(19) United States
(12) Patent Application Publication
(10) Pub. No.: US 2004/0034050 A1
(43) Pub. Date: Feb. 19, 2004

(54) Homo-camptothecin derivatives

(76) Inventor: Li-Xi Yang, (US)

Correspondence Address: COOLEY GODWARD, LLP, 3000 EL CAMINO REAL, 5 PALO ALTO SQUARE, PALO ALTO, CA, 94306, US

(21) Appl. No.: 10/454,525

(22) Filed: Jun. 3, 2003

Related U.S. Application Data
(60) Provisional application No. 60/385,673, filed on Jun. 3, 2002.

(51) Int. Cl. C07D 491/14; A61K 31/496; A61K 31/4745

(52) U.S. Cl. 514/283; 514/253.02; 544/361; 546/48

(57) ABSTRACT

C-20 esters of homo-camptothecin analogues are provided. The compounds are C-20 esters of an oxyalkanoic acid and homo-camptothecin, which are optionally substituted at the 7, 9, 10, 11, and 12 positions of the homo-camptothecin ring. The compounds are useful for treating cancer.

### FIELD OF THE INVENTION

This invention relates to novel homo-camptothecin derivatives that are useful for treating various types of cancer.

### BACKGROUND OF THE INVENTION

Camptothecin (“CPT”), shown below, is an indole alkaloid natural product isolated from the oriental tree Camptotheca acuminata, which has substantial anti-tumor activity in animal models. The compound has a pentacyclic ring system with an asymmetric center in ring E with a 20 S configuration. The pentacyclic ring system includes a pyrrolo [3,4-b] quinoline (rings A, B and C), a conjugated pyridone (ring D), and six membered lactone (ring E) with an 20α-hydroxyl group and a 20-beta ethyl group.

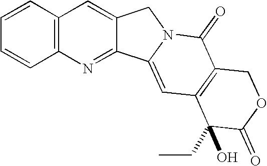

Subsequent studies established that CPT inhibited both DNA and RNA synthesis. Recent research has demonstrated that CPT and CPT analogues interfere with the mechanism of action of the cellular enzyme topoisomerase I, which is important in a number of cellular processes (e.g., DNA replication and recombination, RNA transcription, chromosome decondensation, etc.).

A significant problem with CPT and CPT analogues is the chemical lability of the α-hydroxy lactone functionality. Rapid in situ hydrolysis of the α-hydroxy lactone provides a ring opened carboxylate form which is relatively inactive, as was shown in early clinical studies with CPT sodium.

One solution to the chemical instability of the α-hydroxy lactone group found in CPT is insertion of a carbon atom between the free hydroxy group and the carbonyl carbon of the lactone group to provide a ring expanded β-hydroxy lactone known as homo-Camptothecin (“hCPT”), which is shown below. hCPT is considerably more stable than CPT because of the reduced susceptibility of the lactone carbonyl to nucleophilic attack. Importantly, hCPT possesses substantial anti-proliferative activity in animal studies.

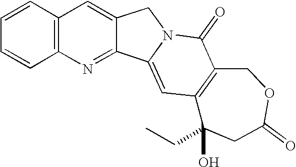

Accordingly there is a need for new analogues of hCPT that may be stable anti-proliferative agents.

### SUMMARY OF THE INVENTION

The present invention satisfies this and other needs by providing compounds which are analogues of hCPT, pharmaceutical compositions of hCPT analogues, methods of using analogues of hCPT and pharmaceutical compositions of hCPT analogues to treat cancer and methods for preparing hCPT analogues.

One aspect of this invention is a compound of the formula (I), below,

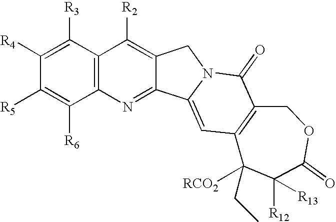

wherein R is R₁—O—(CH₂)ₘ—, m is an integer of 1-10 (preferably 1-5);

R₁ is lower alkyl,

phenyl optionally substituted with from one to five substituents independently selected from the group consisting of halo, lower alkyl, lower alkoxy, hydroxy, cyano, nitro, amino, halogenated lower alkyl, halogenated lower alkoxy, formyl, lower alkyl carbonyl, hydroxycarbonyl, lower alkylcarbonyloxy, benzyloxy, optionally substituted piperidino, lower alkoxycarbonyl, and lower alkylcarbonylamino,

cycloalkyl of 3-7 carbons, optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino,

a fused, 2-, 3-, or 4-ring heterocyclic radical optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino,

1- or 2-naphthyl optionally substituted with from one to four substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino, or

a 5 or 6 membered heterocyclic ring containing one or two nitrogen atoms, which ring is optionally substituted with one or two substituents selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino;

R₂ is hydrogen, halo, lower alkyl, lower alkoxy, hydroxy, RC(O)O (R defined hereinbefore), cyano, nitro, amino, halogenated lower alkyl, halogenated lower alkoxy, hydroxycarbonyl, —C(O)H, lower alkoxycarbonyl, tri lower alkylsilyl, lower alkylcarbonyloxy, lower alkylcarbonylamino, lower alkylcarbonyloxymethyl, substituted vinyl, 1-hydroxy-2-nitroethyl, alkoxycarbonylethyl, aminocarbonyl, mono- or dialkylcarbonyl, alkylcarbonyloymethyl, benzoylmethyl, benzylcarbonyloxymethyl, mono- or di lower alkoyxymethyl, alkoxyiminomethyl, methylpiperazinomethylene, or together with R₃ is —CH₂(NH₂)CH₂CH₂.

R₃ is hydrogen, halo, lower alkyl, lower alkoxy, hydroxy, RC(O)O (R defined hereinbefore) cyano, nitro, amino, halogenated lower alkyl, halogenated lower alkoxy, hydroxycarbonyl, lower alkoxycarbonyl, CH₂NR₇R₈ (where each of R₇ and R₈ is independently H—, alkyl of 1-6 carbons, optionally substituted phenyl, hydroxy lower alkyl, amino lower alkyl, or mono- or dialkylamino lower alkyl, or R₇ and R₈ taken together with —N-represent a cyclic amino-), —C(O)H, CH₂R₉ (where R₉ is lower alkoxy, CN, amino lower alkoxy, mono- or di-lower alkylamino lower alkoxy, lower alkylthio, amino lower alkylthio, or mono- or di-lower alkylamino lower alkylthio), or NR₁₀R₁₁ (where each of R₁₀ and R₁₁ is independently hydrogen, lower alkyl, phenyl, hydroxy lower alkyl, amino lower alkyl, or mono- or di-lower alkyl, or R₁₀ and R₁₁ taken together with —N-represent a cyclic amino), dialkylamino alkyl, lower alkylcarbonyloxy, lower alkylcarbonylamino or trialkysilyl ehtylene;

R₄ is hydrogen, halo, lower alkyl, lower alkoxy, hydroxy, RC(O)O (R defined hereinbefore) cyano, nitro, amino, amino lower alkyl, halogenated lower alkyl, halogenated lower alkoxy, hydroxycarbonyl, lower alkoxycarbonyl, carbamoyloxy, lower alkylcarbonyloxy, or lower alkylcarbonylamino, or R₄ together with R₅ is methylenedioxy or ethylenedioxy;

R₅ is hydrogen, halo, lower alkyl, lower alkoxy, hydroxy, RC(O)O (R defined hereinbefore), cyano, nitro, amino, halogenated lower alkyl, halogenated lower alkoxy, hydroxycarbonyl, lower alkoxycarbonyl, lower alkylcarbonyloxy, or lower alkylcarbonylamino or together with R₄ is methylenedioxy or ethylenedioxy;

R₆ is hydrogen, halo, lower alkyl, lower alkoxy, hydroxy, RC(O)O (R defined hereinbefore), cyano, nitro, amino, halogenated lower alkyl, halogenated lower alkoxy, hydroxycarbonyl, lower alkoxycarbonyl, lower alkylcarbonyloxy, or lower alkylcarbonylamino; and

R₁₂ and R₁₃ are independently hydrogen, lower alkyl, cyano, hydroxycarbonyl, lower alkoxycarbonyl, cycloalkyl, alkylcarbonylamino, 1-napthyl, 2-napthyl or phenyl.

Another aspect of the invention is a pharmaceutical composition useful for treating cancer in a warm-blooded animal, which composition comprises compound of the invention as defined herein in combination with a pharmaceutically acceptable excipient.

Another aspect of this invention is a method for treating cancer in a warm-blooded animal, which method comprises administering a therapeutically effective amount of a compound of the invention as defined herein. The compound is administered in a therapeutically effective dose by appropriate administration, e.g., orally, topically, or parenterally.

Another aspect of this invention is process for preparing compounds of this invention by reacting homo-camptothecin (hCPT) or a hCPT analogue with a compound of the formula R—C(O)X, wherein R is R₁—O—(CH₂)m, R₁ is as defined herein, m is an integer of 1-10, and X is e.g., bromide, chloride, hydroxy, alkoxy of 1-11 carbons (e.g., —O(CH₂)ₘ CH₃ where m is an integer of 1-10) or R—C(O)O—(R is defined hereinbefore).

Other aspects of this invention will be apparent to one of skill in the art by reviewing the ensuing specification.

### DETAILED DESCRIPTION

### Overview

In general this invention can be viewed as a (C-20) ester of hCPT or a hCPT analogue (preferably, a (20S) ester of hCPT or a hCPT analogue). The hCPT has a hydroxy at the 20 position, which may be esterified in accordance with the process of this invention to form the corresponding ester in good yield. The resulting ester is unique in that it has an electronegative entity in the chain, which is believed to aid in stabilizing the E ring of the homo-camptothecin molecule. The novel compounds of the invention are active against tumors in mice and are generally well tolerated. They are useful for treating various types of cancer and can be formulated to prepare pharmaceutical preparations for oral, topical, or parenteral administration.

While not wishing to be bound by any particular mechanism of action or theoretical explanation of how the compounds work, it is believed that the C-20 esters exert their effect in part by stabilizing the E ring of the hCPT molecule. The esters may accomplish this through steric hindrance by preventing enzymatic access to the E ring, through the presence of an electron-withdrawing group in the ester chain, i.e. an oxygen atom, and through facilitating the hydrogen-binding or Van Der Waals forces of the E ring end of the hCPT molecule with the enzyme to inhibit binding and thus enzyme activity, which may sever the E ring.

### Definitions

The term “hCPT” is an abbreviation for homo-camptothecin, which may be synthesized from CPT by methods known in the art. The chemical formula of homo-camptothecin and its numbering system are as follows:

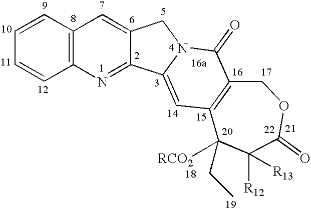

The compound has a hydroxy at the 20-position(s) that may be esterified to make the compounds of this invention. For purposes of this application the bond at 20 for the oxygen link will be designated with a straight line, as will the bond for the ethyl group. The numbering system for hCPT is based on the system for CPT as set forth in U.S. Pat. No. 6,350,756 issued to Yang, et. al. Feb. 6, 2002, which patent in its entirety is incorporated herein by reference.

The term “alkyl” refers to a monovalent, saturated aliphatic hydrocarbon radical having the indicated number of carbon atoms. For example, a “C1-6 alkyl” or an “alkyl of 1-6 carbons” or “Alk 1-6 would refer to any alkyl group containing one to six carbons in the structure. “C1-20 alkyl” refer to any alkyl group having one to twenty carbons. Alkyl may be a straight chain (i.e., linear) or a branched chain. Lower alkyl refers to an alkyl of 1-6 carbons. Representative examples lower alkyl radicals include methyl, ethyl, n-propyl, n-butyl, n-pentyl, n-hexyl, isopropyl, isobutyl, isopentyl, amyl, sec-butyl, tert-butyl, tert-pentyl and the like. Higher alkyl refers to alkyls of seven carbons and above. These include n-heptyl, n-octyl, n-nonyl, n-decyl, n-dodecyl, n-tetradecyl, n-hexadecyl, n-octadecyl, n-eicosyl, and the like, along with branched variations thereof. The radical may be optionally substituted with substituents at positions that do not significantly interfere with the preparation of compounds falling within the scope of this invention and that do not significantly reduce the efficacy of the compounds. The alkyl is optionally substituted with one to five substituents independently selected from the group consisting of halo, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

The term “alkoxy” refers to a monovalent radical of the formula RO—, where R is an alkyl as defined herein. Lower alkoxy refers to an alkoxy of 1-6 carbon atoms, with higher alkoxy is an alkoxy of seven or more carbon atoms. Representative lower alkoxy radicals include methoxy, ethoxy, n-propoxy, n-butoxy, n-pentyloxy, n-hexyloxy, isopropoxy, isobutoxy, isopentyloxy, amyloxy, sec-butoxy, tert-butoxy, tert-pentyloxy, and the like. Higher alkoxy radicals include those corresponding to the higher alkyl radicals set forth herein. The radical may be optionally substituted with substituents at positions that do not significantly interfere with the preparation of compounds falling within the scope of this invention and that do not significantly reduce the efficacy of the compounds. The alkyl is optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

The term “cycloalkyl” refers to a monovalent, alicyclic, saturated hydrocarbon radical having three or more carbons forming the ring. While known cycloalkyl compounds may have up to 30 or more carbon atoms, generally there will be three to seven carbons in the ring. The latter include, for example, cyclopropyl, cyclobutyl, cyclopentyl, cyclohexyl, and cycloheptyl. The radical may be optionally substituted with substituents at positions that do not significantly interfere with the preparation of compounds falling within the scope of this invention and that do not significantly reduce the efficacy of the compounds. The cycloalkyl is optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

The term “hydroxycarbonyl” is a monovalent radical having the formula —C(O)OH.

The term “lower alkoxycarbonyl” is a monovalent radical having the formula —C(O)OAlk, where Alk is lower alkyl.

The term “lower alkylcarboxyloxy” is a monovalent radical having the formula —OC(O)Alk, where Alk is lower alkyl.

The term “lower alkylcarbonylamino” is a monovalent radical having the formula —NHC(O)Alk, where Alk is lower alkyl.

A “halo” substitutent is a monovalent halogen radical chosen from chloro, bromo, iodo, and fluoro. A “halogenated” compound is one substituted with one or more halo substituent.

A “1-naphthyl” or “2-naphthyl” is a radical formed by removal of a hydrogen from the 1- or 2-position of a naphthalene structure, respectively. It is optionally substituted with from one to four substituents independently selected from the group consisting of halo, lower alkyl, lower alkoxy, hydroxy, cyano, nitro, amino, halogenated lower alkyl, formyl, halogenated lower alkoxy, hydroxycarbonyl, lower alkoxycarbonyl, lower alkylcarbonyloxy, and lower alkylcarbonylamino.

A “phenyl” is a radical formed by removal of a hydrogen from a benzene ring. The phenyl is optionally substituted with from one to five substituents independently selected from the group consisting of halo, lower alkyl, lower alkoxy, hydroxy, cyano, nitro, amino, halogenated lower alkyl, halogenated lower alkoxy, carbonyl, hydroxycarbonyl, lower alkylcarbonyloxy, benzyloxy, optionally substituted piperidino, lower alkoxycarbonyl, and lower alkylcarbonylamino.

A “cyclic amino” is a monovalent radical of saturated 5-, 6-, or 7-membered cyclic amine ring having no more than one additional hetero atom such as nitrogen, oxygen, or sulfur. Representative examples include, e.g., 1-pyrrolidino, 1-piperidino, morpholino, piperazino, and the like. These may be substituted or unsubstituted. If substituted, generally they will have no more than 2 substituents chosen from lower alkyl, lower cycloalkyl, hydroxy lower alkyl, phenyl (substituted or unsubstituted), benzyl (substituted or unsubstituted), aminocarbonylmethyl, lower alkylaminocarbonylmethyl, amino, mono- or di-lower alkylamino, or cyclic amino.

A “carbamoyloxy” is a monovalent radical of the formula R₁₃R₁₄NC(O)O— (i.e. an aminocarbonyloxy) where R₁₃ and R₁₄ together form a cyclic amino with the nitrogen atom, or each of R₁₃ and R₁₄ is independently hydrogen, lower alkyl, hydroxy lower alkyl, hydroxy lower alkyl, amino lower alkyl, lower cycloalkyl, phenyl (substituted or unsubstituted), or benzyl (substituted or unsubstituted). Examples include aminocarbonyloxy, methylaminocarbonyloxy, dimethyl aminocarbonyloxy, [4-(1-piperidino)-1-piperidino] carbonyloxy, 1-morpholinocarbonyloxy, 1-pyrrolidinyl, 1-piperazinecarbonyloxy, and others delineated herein.

A “5-membered heterocyclic ring” is a monovalent radical of a 5-member closed ring containing carbon and at least one other element, generally nitrogen, oxygen, or sulfur and may be fully saturated, partially saturated, or unsaturated (i.e., aromatic in nature). Generally the heterocycle will contain no more than two hetero atoms. Representative examples of unsaturated 5-membered heterocycles with only one hetero atom include 2- or 3-pyrrolyl, 2- or 3-furanyl, and 2- or 3-thiophenyl. Corresponding partially saturated or fully saturated radicals include 3-pyrrolin-2-yl, 2- or 3-pyrrolidinyl, 2- or 3-tetrahydrofuranyl, and 2- or 3-tetrahydrothiophenyl. Representative unsaturated 5-membered heterocyclic radicals having two hetero atoms include imidazolyl, oxazolyl, thiazolyl, pyrazolyl, and the like. The corresponding fully saturated and partially saturated radicals are also included. The heterocyclic radical is bonded through an available carbon atom in the heterocyclic ring. The radical may be optionally substituted with substituents at positions that do not significantly interfere with the preparation of compounds falling within the scope of this invention and that do not significantly reduce the efficacy of the compounds. The ring is optionally substituted with one or two substituents selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy and lower alkylcarbonylamino.

A “6-membered heterocyclic ring” is a monovalent radical of a 6-member closed ring containing carbon and at least one other element, generally nitrogen, oxygen, or sulfur and may be fully saturated, partially saturated, or unsaturated (i.e., aromatic in nature). Generally the heterocycle will contain no more than two hetero atoms. Representative examples of unsaturated 6-membered heterocycles with only one hetero atom include 2-, 3-, or 4-pyridinyl, 2H-pyranyl, and 4H-pryanyl. Corresponding partially saturated or fully saturated radicals include 2-, 3-, or 4-piperidinyl, 2-, 3-, or 4-tetrahydropyranyl and the like. Representative unsaturated 6-membered heterocyclic radicals having two hetero atoms include 3- or 4-pyridazinyl, 2-, 4-, or 5-pyrimidinyl, 2-pyrazinyl, and the like. The corresponding fully saturated and partially saturated radicals are also included, e.g., 2-piperazine. The heterocyclic radical is bonded through an available carbon atom in the heterocyclic ring. The radical may be optionally substituted with substituents at positions that do not significantly interfere with the preparation of compounds falling within the scope of this invention and that do not significantly reduce the efficacy of the compounds. The ring is optionally substituted with one or two substituents selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

A “fused 2-, 3-, or 4-ring heterocyclic radical” is polynuclear in that the adjacent rings share a pair of atoms, generally carbon atoms. At least one of the rings will be heterocyclic in that it will have a noncarbon atom such as nitrogen, oxygen, or sulfur. The ring system may contain from 9 to 18 atoms. A 2-ring heterocyclic system will generally have 9 or 10 atoms included in the ring. Examples of such a 2-ring system include quinoline, isoquinoline, purine, indolizine, 4H-quinolizine, 3H-pyrrolizine, coumaran, coumarin, isocoumarin, 4-methylcoumarin, 3-chloro-H-methylcoumarin, chromone, benzofuran, benzothiophene, benzothiazole, indole, and the like. A 3-ring system will generally have 12 to 14 atoms included in the ring. Examples of such a 3-ring system include carbazole, acridine, and the like. A 4-ring fused system will generally have 16 to 18 atoms included in the chain. Examples of such a 4-ring system include isothebaine and the like. The ring is bonded through a carbon in the ring system. The radical may be optionally substituted with substituents at positions that do not significantly interfere with the preparation of compounds falling within the scope of this invention and that do not significantly reduce the efficacy of the compounds. The radical is optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

Other chemical terms are given their standard meaning as understood by one of skill in the art with guidance from standard texts and dictionaries.

The term “MTD” is the abbreviation for maximum tolerated dose.

The term “nM” is the abbreviation for nanomolar.

The term “ip” is the abbreviation for intraperitoneal.

### Compounds of the Invention

One aspect of this invention is a compound of the formula

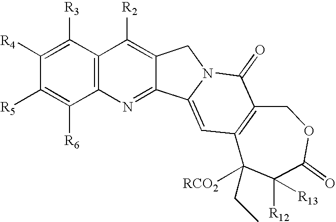

wherein R is R₁—O—(CH₂)ₘ—, m is an integer of 1-10 (preferably 1-5, particularly 1);

R₁ is lower alkyl,

phenyl optionally substituted with from one to five substituents independently selected from the group consisting of halo, lower alkyl, lower alkoxy, hydroxy, cyano, nitro, amino, halogenated lower alkyl, halogenated lower alkoxy, formyl, lower alkyl carbonyl, hydroxycarbonyl, lower alkylcarbonyloxy, benzyloxy, optionally substituted piperidino, lower alkoxycarbonyl, and lower alkylcarbonylamino,

cycloalkyl of 3-7 carbons, optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino,

a fused, 2-, 3-, or 4-ring heterocyclic radical optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino,

1- or 2-naphthyl optionally substituted with from one to four substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino or

a 5 or 6 membered heterocyclic ring containing one or two nitrogen atoms, which ring is optionally substituted with one or two substituents selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino;

R₂ is hydrogen, halo, lower alkyl, lower alkoxy, hydroxy, RC(O)O (R defined hereinbefore), cyano, nitro, amino, halogenated lower alkyl, halogenated lower alkoxy, hydroxycarbonyl, —C(O)H, lower alkoxycarbonyl, tri lower alkylsilyl, lower alkylcarbonyloxy, lower alkylcarbonylamino, lower alkylcarbonyloxymethyl, substituted vinyl, 1-hydroxy-2-nitroethyl, alkoxycarbonylethyl, aminocarbonyl, mono- or di-alkylcarbonyl, alkylcarbonyloymethyl, benzoylmethyl, benzylcarbonyloxymethyl, or mono- or di lower alkoyxymethyl;

R₃ is hydrogen, halo, lower alkyl, lower alkoxy, hydroxy, RC(O)O (R defined hereinbefore) cyano, nitro, amino, halogenated lower alkyl, halogenated lower alkoxy, hydroxycarbonyl, lower alkoxycarbonyl, CH₂NR₇R₈ (where each of R₇ and R₈ is independently H—, alkyl of 1-6 carbons, optionally substituted phenyl, hydroxy lower alkyl, amino lower alkyl, or mono- or dialkylamino lower alkyl, or R₇ and R₈ taken together with —N—represent a cyclic amino-), —C(O)H, CH₂R₉ (where R₉ is lower alkoxy, CN, amino lower alkoxy, mono- or di-lower alkylamino lower alkoxy, lower alkylthio, amino lower alkylthio, or mono- or di-lower alkylamino lower alkylthio), or NR₁₀R₁₁ (where each of R₁₀ and R₁₁ is independently hydrogen, lower alkyl, phenyl, hydroxy lower alkyl, amino lower alkyl, or mono- or di-lower alkyl, or R₁₀ and R₁₁ taken together with —N— represent a cyclic amino), dialkylamino alkyl, lower alkylcarbonyloxy, or lower alkylcarbonylamino;

R₄ is hydrogen, halo, lower alkyl, lower alkoxy, hydroxy, RC(O)O (R defined hereinbefore) cyano, nitro, amino, amino lower alkyl, halogenated lower alkyl, halogenated lower alkoxy, hydroxycarbonyl, lower alkoxycarbonyl, carbamoyloxy, lower alkylcarbonyloxy, or lower alkylcarbonylamino, or R₄ together with R₅ is methylenedioxy or ethylenedioxy;

R₅ is hydrogen, halo, lower alkyl, lower alkoxy, hydroxy, RC(O)O (R defined hereinbefore), cyano, nitro, amino, halogenated lower alkyl, halogenated lower alkoxy, hydroxycarbonyl, lower alkoxycarbonyl, lower alkylcarbonyloxy, or lower alkylcarbonylamino or R₅ together with R₄ is methylenedioxy or ethylenedioxy;

R₆ is hydrogen, halo, lower alkyl, lower alkoxy, hydroxy, RC(O)O (R defined hereinbefore), cyano, nitro, amino, halogenated lower alkyl, halogenated lower alkoxy, hydroxycarbonyl, lower alkoxycarbonyl, lower alkylcarbonyloxy, or lower alkylcarbonylamino; and

R₁₂ and R₁₃ are independently hydrogen, lower alkyl, cyano, hydroxycarbonyl, lower alkoxycarbonyl, cycloalkyl, alkylcarbonylamino, 1-napthyl, 2-napthyl or phenyl. Preferred aspects of the invention are set forth hereinafter in the claims and specification.

A preferred aspect is a compound of formula (I) wherein m is 1, R₁ is phenyl optionally substituted with one to three substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, and benzyloxy; and each of R₂, R₃, R₄, R₅, R₆, R₁₂ and R₁₃ is H or R₂, R₃, R₄, R₅, R₆, R₁₂ and R₁₃ are as described in the further discussion of preferred aspects as set forth hereinafter. In this subgroup, another preferred aspect is a compound wherein R₁ is phenyl optionally substituted with one to three substituents independently selected from lower alkyl, halo, halogenated lower alkoxy, and lower alkoxy, particularly phenyl optionally substituted with one to three halo substituents. Also included are the pharmaceutically acceptable salts of these compounds. Pharmaceutically-acceptable salts are those salts formed by reacting an organic or inorganic acid with a compound represented by formula (I) where there is a reactive base (e.g., an available nitrogen). Suitable salts include, e.g., the acetate, hydrochloride, sulfate, phosphate, and the like. Others will be apparent to one of skill in the art by consulting standard sources such as Remington's mentioned herein.

Another preferred aspect is a compound wherein m is 1; each of R₂, R₃, R₄, R₅, R₆, R₁₂ and R₁₃ is H (or the preferences described hereinafter); and R₁ is a fused, 2-ring heterocyclic system., e.g., a compound wherein R₁ is represented by the formulas

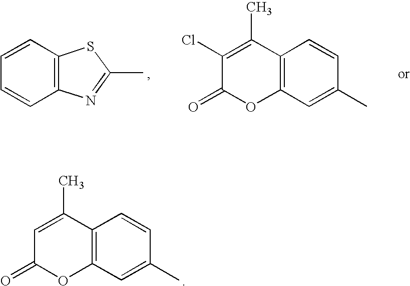

The names of these radicals are 2-benzothiazolyl, 3-chloro-4-methylcoumarin-7-yl, and 4-methylcoumarin-7-yl, respectively.

Another aspect is a compound of wherein m is 1: each of R₂, R₃, R₄, R₅, R₆, R₁₂ and R₁₃ is H (or the preferences discussed hereinafter); and R₁ is 1- or 2-naphthyl optionally substituted with from one to, four substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy and lower alkylcarbonylamino. Preferably R₁ is 2-naphthyl.

Another aspect is a compound wherein m is 1, each of R₂, R₃, R₄, R₅, R₆, R₁₂ and R₁₃ is H (or the preferences described hereinafter), and R₁ is 4-formylphenyl or

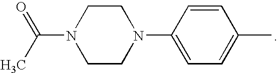

The latter radical is named 4-[4-acetylpiperazin-1-yl)phenyl.

Another aspect is a compound wherein m is an integer of 2-4; each of R₂, R₃, R₄, R₅, R₆, R₁₂ and R₁₃ is H (or the preferences described hereinafter); and R₁ is phenyl optionally substituted with from one to five substituents independently selected from the group consisting of halo, lower alkyl, lower alkoxy, hydroxy, cyano, nitro, amino, halogenated lower alkyl, halogenated lower alkoxy, carbonyl, hydroxycarbonyl, lower alkoxycarbonyl, benzyloxy, lower alkylcarbonyloxy and lower alkylcarbonylamino.

Other aspects of the invention include compounds as described hereinbefore, but where each R₂, R₃, R₄, R₅, R₆, R₁₂ and R₁₃ may be a substituent other than only hydrogen. These include, for example, the preferred subgroups set forth hereinafter:

The compound of formula (I) wherein R₆ is hydrogen, particularly a compound wherein R₄ and R₅ together are methylenedioxy and wherein R₂ is hydrogen. Of these the compounds particular interest are those where R₃ is nitro, amino, methyl, chloro, cyano, acetoxy, or acetylamino.

A compound of formula (I), wherein each of R₅ and R₆ is hydrogen, especially those wherein R₃ is hydrogen; R₂ is (3-chloro-n-propyl)dimethylsilyl, tert-butyldimethylsilyl, acetoxymethyl, cyano, formylethenyl, ethoxycarbonyl-ethenyl, cyanoethenyl, 2,2-dicyanoethenyl, (2-cyano-2-ethoxycarbony)ethenyl, ethoxycarbonylethyl, methyl, ethyl, or n-propyl; and R₄ is hydroxy, acetoxy, amino, nitro, cyano, chloro, bromo, fluoro, lower alkyl, higher alkyl, lower alkoxy, carbamoyloxy, or formyl. Of these, the compounds wherein R₂ is ethyl and R₄ is carbamoyloxy are of further interest. Carbamoyloxy substituents that are preferred include 1-pyrazinylcarbonyloxy, 4-(i-propylaminocarbonylmethyl) pyrazin-1-yl-carbonyloxy, or 4-(1-piperidio)-1-piperidinocarbonyloxy.

The compound of formula (I), wherein each of R₂, R₅, and R₆ is hydrogen, for example, those wherein R₃ is amino, nitro, cyano, halo, OH, lower alkylamino, di-lower alkylamino, lower alkyl, lower alkoxy, 1-piperidino, 1-mopholino, aminomethyl, lower alkylaminomethyl, cycloalkylaminomethyl, di-lower alkylaminomethyl, cyclic aminomethyl, acetoxy, acetylamino, lower alkoxymethyl, omega hydroxy lower alkylaminomethyl, cyanomethyl and R₄ is hydroxy, acetoxy, cyano, nitro, amino, halo, formyl, lower alkoxy, carbamoyloxy.

A compound wherein each of R₂, R₃, R₅ and R₆ is hydrogen and R₄ is —OC(O)Alkyl₁₋₂₀.

A compound of Formula (I) wherein R₁₂ is lower alkyl, cyano, hydroxycarbonyl, lower alkoxycarbonyl, cycloalkyl, alkylcarbonylamino, 1-napthyl, 2-napthyl or phenyl and R₁₃ is hydrogen. Preferably, R₁₂ is lower alkyl, cycloalkyl, 1-napthyl, 2-napthyl or phenyl, more preferably, R₁₂ is lower alkyl or phenyl.

A compound of Formula (I) wherein R₁₂ is hydrogen and R₁₃ is lower alkyl, cyano, hydroxycarbonyl, lower alkoxycarbonyl, cycloalkyl, alkylcarbonylamino, 1-napthyl, 2-napthyl or phenyl. Preferably, R₁₃ is lower alkyl, cycloalkyl, 1-napthyl, 2-napthyl or phenyl, more preferably, R₁₃ is lower alkyl or phenyl.

A compound of Formula (I) wherein R₁₂ and R₁₃ are hydrogen, lower alkyl, cyano, hydroxycarbonyl, lower alkoxycarbonyl, cycloalkyl, alkylcarbonylamino, 1-napthyl, 2-napthyl or phenyl. Preferably, R₁₂ and R₁₃ are hydrogen, lower alkyl, cycloalkyl, 1-napthyl, 2-napthyl or phenyl, more preferably, R₁₂ and R₁₃ are hydrogen, lower alkyl or phenyl.

### Pharmaceutical Composition of the Invention

This aspect of the invention is a pharmaceutical composition useful for treating cancer in a warm-blooded animal, which composition comprises compound of the invention as defined herein in combination with a pharmaceutically acceptable excipient. The composition is prepared in accordance with known formulation techniques to provide a composition suitable for oral, topical, transdermal, rectal, by inhalation, parenteral (intravenous, intramuscular, or intraperitoneal) administration, and the like. Detailed guidance for preparing compositions of the invention are found by reference to the 18^{th} or 19^{th} Edition of Remington's Pharmaceutical. Sciences, Published by the Mack Publishing Co., Easton, Pa. 18040. The pertinent portions are incorporated herein by reference.

Unit doses or multiple dose forms are contemplated, each offering advantages in certain clinical settings. The unit dose would contain a predetermined quantity of active compound calculated to produce the desired effect(s) in the setting of treating cancer. The multiple dose form may be particularly useful when multiples of single doses, or fractional doses, are required to achieve the desired ends. Either of these dosing forms may have specifications that are dictated by or directly dependent upon the unique characteristic of the particular compound, the particular therapeutic effect to be achieved, and any limitations inherent in the art of preparing the particular compound for treatment of cancer.

A unit dose will contain a therapeutically effective amount sufficient to treat cancer in a subject and may contain from about 1.0 to 1000 mg of compound, for example about 50 to 500 mg.

The compound will preferably be administered orally in a suitable formulation as an ingestible tablet, a buccal tablet, capsule, caplet, elixir, suspension, syrup, trouche, wafer, lozenge, and the like. Generally, the most straightforward formulation is a tablet or capsule (individually or collectively designated as an “oral dosage unit”). Suitable formulations are prepared in accordance with a standard formulating techniques available that match the characteristics of the compound to the excipients available for formulating an appropriate composition. A tablet or capsule will contain about 50 to about 500 mg of a compound of Formula (I).

The form may deliver a compound rapidly or may be a sustained-release preparation. The compound may be enclosed in a hard or soft capsule, may be compressed into tablets, or may be incorporated with beverages, food or otherwise into the diet. The percentage of the final composition and the preparations may, of course, be varied and may conveniently range between 1 and 90% of the weight of the final form, e.g., tablet. The amount in such therapeutically useful compositions is such that a suitable dosage will be obtained. Preferred compositions according to the current invention are prepared so that an oral dosage unit form contains between about 5 to about 50% by weight (%w) in dosage units weighing between 50 and 1000 mg.

The suitable formulation of an oral dosage unit may also contain: a binder, such as gum tragacanth, acacia, corn starch, gelatin; sweetening agents such as lactose or sucrose; disintegrating agents such as corn starch, alginic acid and the like; a lubricant such as magnesium stearate; or flavoring such a peppermint, oil of wintergreen or the like. Various other material may be present as coating or to otherwise modify the physical form of the oral dosage unit. The oral dosage unit may be coated with shellac, a sugar or both. Syrup or elixir may contain the compound, sucrose as a sweetening agent, methyl and propylparabens as a preservative, a dye and flavoring. Any material utilized should be pharmaceutically-acceptable and substantially non-toxic. Details of the types of excipients useful may be found in the nineteenth edition of “Remington: The Science and Practice of Pharmacy,” Mack Printing Company, Easton, Pa. See particularly chapters 91-93 for a fuller discussion.

A compound may be administered parenterally, e.g., intravenously, intramuscularly, intravenously, subcutaneously, or interperitonieally. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of the compound may be prepared in suitable diluents such as water, ethanol, glycerol, liquid polyethylene glycol(s), various oils, and/or mixtures thereof, and others known to those skilled in the art.

The pharmaceutical forms suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

In some cases, e.g., where a compound of the invention is quite water insoluble, it may be useful to provide liposomal delivery. The system restrains the compound of the invention by incorporating, encapsulating, surrounding, or entrapping the compound of the invention in, on, or by lipid vesicles or liposomes, or by micelles.

Usefully, the compound of the invention is solubilized in liposomes. The liposomes may include, for example, lipids such as cholesterol, phospholipids, or micelles comprised of surfactant such as, for example, sodium dodecylsylfate, octylphenolpolyoxyethylene glycol, or sorbitan mono-oleate. Typically, the compound of the invention binds to the lipid bilayer membrane of the liposome with high affinity. The liposome bound prodrug can preferably intercalate between the acyl chains of the lipid. The lactone ring of the homo-camptothecinderivative, membrane-bound compound of the invention is thereby removed from the aqueous environment inside and outside of the liposome and further protected from hydrolysis. Since the liposome-bound drug is protected from hydrolysis, the antitumor activity of the drug is preserved. For a compound of the invention which has a lower affinity for the liposome membrane and thus disassociates from the liposome membrane to reside in the interior of liposome, the pH of the interior of the liposomes may be reduced thereby preventing hydrolysis of such compound of the invention.

A useful group of liposomal delivery systems which may be used in accordance with the present invention include those described in U.S. Pat. Nos. 5,552,156 and 5,736,156, which are herein incorporated in their entireties by reference. Other liposomal delivery systems which may be employed in accordance with the present invention include liposomes containing active agents aggregated with lipids or surfactants as described in U.S. Pat. Nos. 5,827,533 and 5,882,679; lipid vesicles formed with alkyl ammonium fatty acid salts as described in U.S. Pat. No. 5,874,105; liposomes for encapsulating active agent dry powder compositions as described in U.S. Pat. No. 5,783,211; liposomal drug delivery systems for topical patches as described in U.S. Pat. No. 5,718,914; the liposomes described in U.S. Pat. No. 5,631,237; the liposome and lipid complex compositions described in -U.S. Pat. Nos. 5,549,910 and 5,077,057; the liposomes used for sustained release of steroidal drugs as described in U.S. Pat. No. 5,043,165; the liposomes described in U.S. Pat. No. 5,013,556; and the liposomes described in U.S. Pat. No. 4,663,161; all of which are herein incorporated in their entireties by reference.

Unilamellar liposomes, also referred to as single lamellar vesicles, are spherical vesicles comprised of one lipid bilayer membrane which defines a closed compartment. The bilayer membrane is composed of two layers of lipids; an inner layer and an outer layer. The outer layer of lipid molecules are oriented with their hydrophilic head portions toward the external aqueous environment and their hydrophobic tails pointed downward toward interior of the liposome. The inner layer of lipid lays directly beneath the outer layer; the lipids are oriented with their heads facing the aqueous interior of the liposome and their tails toward the tails of outer layer of lipid.

Multilamellar liposomes, also referred to as multilamellar vesicles, are composed of more than one lipid bilayer membrane, which membranes define more than one closed compartment. The membranes are concentrically arranged so that the different membranes are separated by compartments much like an onion skin.

Thus, some or all of the compound of the invention is located in one or more of the compartments of a liposome or micelle, or the compound of the invention is bound to the membrane of the liposome. Where a compound is bound to a lipid membrane, at least the lactone ring of some or all of the compound of the invention binds to the lipid membrane of the liposome, and where the liposome contains more than one bilayer membrane the compound of the invention is bound to at least 1 membrane. Those compounds of the invention that have a high affinity for such membrane tend to remain bound to the membrane. Those compounds of the invention with a low affinity for liposome membrane, will at least partially disassociate from the liposome membrane and reside in the liposome compartment.

Micelles as defined herein are spherical receptacles comprised of a single, monolayer membrane which defines a closed compartment and the membrane is comprised of surfactant molecules oriented so that the hydrocarbon tails are oriented toward the compartment and the polar head portions are oriented toward the external aqueous environment. The compounds of the invention, when associated with micelles, are either in the compartment, bound to the micelle membrane, or bound to the outside surface of the micelle.

Liposomes have been used successfully to administer medications to cancer patients, and have been shown to be useful clinically in the delivery of anticancer drugs such as doxorubicin, daunorubicin, and cis-platinum complexes. Forssen et al., Cancer Res. 1992, 52: 3255-3261; Perex-Soler, et al., Cancer Res. 1990, 50: 4260-4266; and, Khokhar, et al., J. Med. Chem. 1991, 34: 325-329, all of which are incorporated herein in their entireties by reference.

Similarly, micelles have also been used to deliver medications to patients, (Broden et al., Acta Pharm Suec. 19: 267-284 (1982)) and micelles have been used as drug carriers and for targeted drug delivery, (D. D. Lasic, Nature 335: 279-280 (1992); and, Supersaxo et al., Pharm Res. 8: 1280-1291 (1991)), including cancer medications, (Fung et al., Biomater. Artif. Cells. Artif. Organs 16: 439 et seq. (1988); and Yokoyama et al., Cancer Res. 51: 3229-3236 (1991)), al of which are incorporated herein in their entirefies by reference.

The liposomes and/or micelles containing the compound of the invention can be administered to a cancer patient, typically intravenously. The liposomes and/or micelles are carried by the circulatory system to the cancer cells where the membrane of the vesicle fuses to the membrane of the cancer cell thereby releasing the compound of the invention to the cancer cell, or where the liposomes and/or micelles to be taken up by the cancer cells, the compound of the invention diffuses from the liposomes and/or micelles to be taken up by the cancer cells.

Any lipid mixture of lipids which forms liposomes and/or micelles is suitable for use in the present invention. Phosphatidylcholines, including, for example, L-.alpha.-dimyristoylphosphatidylcholine (DPMC), 1-.alpha.-dipalmitoylphosphatidylcholine (DPPC) and L-.alpha.-distearoylphosphatidylcholine (DSPC) are suitable. Also, phosphatidylglycerols, including, for example, L-.alpha.-dimyristoylphosphatidylglycerol (DMPG) are suitable. The DMPC and DMPG are both fluid phase at 37, for example, L-.alpha.-dimyristoylphosphatidylglycerol (DMPG) are suitable. The DMPC and DMPG are both fluid phase at 37° C., while DSPC is solid phase at 37° C. Since the presence of negatively charged lipid in the liposome membrane causes the liposomes to repel each other, small amounts, such as, for example about 10%, of an negatively charged lipid, such as distearolphosphotidylglycerol (DSPG), may be incorporated in to the DSPC liposomes. Other suitable phospholipids include: phosphatidyl-ethanolamines, phosphatidylinositols, and phosphatidic acids containing lauric, myristic, palmitic, palmitoleic, stearic, oleic, linoleic, arachidonic, behenic and lignoceric acid. Another suitable lipid includes cholesterol.

U.S. Pat. No. 6,096,336 provides further guidance for preparing liposomal compositions useful in this invention and is incorporated herein by reference.

### Method of Treatment of the Invention

Another aspect of this invention is a method for treating cancer in a warm-blooded animal, which method comprises administering a therapeutically effective amount of a compound of the invention as defined herein. A compound useful in this invention is administered to an appropriate subject in need of these compounds in a therapeutically effective dose by a medically acceptable route of administration such as orally, parentally (e.g., intramuscularly, intravenously, subcutaneously, interperitoneally), transdermally, rectally, by inhalation and the like.

The term cancer is to be considered in the broadest general definition as a malignant neoplasm, an abnormal mass of tissue, the growth of which exceeds and is uncoordinated with that of normal tissues and persists in the same excessive manner after cessation of the stimuli that evoked the change. It might be added that the abnormal mass is purposeless, preys on the host, and is virtually autonomous. A cancer can also be considered as a malignant tumor. A further discussion of neoplasia is found at “Robbins Pathologic Basis of Disease,” Sixth Edition, by R. S. Cotran, V. Kumar, and T. Collins, Chapter 8 (W. B. Saunders Company). This information from Chapter 8 is incorporated herein by reference. The following Table A provides examples of the types of cancers, i.e., malignant tumors or neoplasia that may be treated by administering a compound of this invention.

TABLE A

| Tissue of Origin | Malignant |
|---|---|
| Composed of One Parenchymal Cell Type | |
| Mesenchymal tumors | |
| Connective tissue and derivatives | Fibrosarcoma |
| | Liposarcoma |
| | Chondrosarcoma |
| | Osteogenic sarcoma |
| Endothelial and related tissues | |
| Blood vessels | Angiosarcoma |
| Lymph vessels | Lymphangiosarcoma |
| Synovium | Synovial sarcoma |
| Mesothelium | Mesothelioma |
| Brain coverings | Invasive meningioma |
| Blood cells and related cells | |
| Hematopoietic cells | Leukemias |
| Lymphoid tissue | Malignant lymphomas |
| Muscle | |
| Smooth | Leiomyosarcoma |
| Straited | Rhabdomyosarcoma |
| Epthelial tumors | |
| Stratified squamous | Squamous cell or |
| | epidermoid carcinoma |
| Basal cells of skin or adnexa | Basal cell carcinoma |
| Epithelial lining | |
| Glands or ducts | Adenocarcinoma |
| | Papillary carcinoma |
| | Cystadenocarcinoma |
| Respiratory passages | Bronchogenic carcinoma |
| | Bronchial adenoma |
| | (carcinoid) |
| Neuroectoderm | Malignant melanoma |
| Renal epithelium | Renal cell carcinoma |
| Liver cells | Hepatocellular carcinoma |
| Urinary tract epithelium (transitional) | Transitional cell carcinoma |
| Placental epithelium (trophoblast) | Choriocarcinoma |
| Testicular epithelium (germ cells) | Seminoma |
| | Embryonal carcinoma |
| More Than One Neoplastic Cell-Mixed | |
| Tumors, Usually Derived From One Germ | |
| Layer | |
| Salivary glands | Malignant mixed tumor of |
| | salivary gland origin |
| Breast | Malignant cystosarcoma |
| | phyllodes |
| Renal anlage | Wilms tumor |
| More Than One Neoplastic Cell Type | |
| Derived From More Than One Germ | |
| Layer-Teratogenous | |
| Totipotential cells in gonads or in | Immature teratoma, |
| embryonic rests | teratocarcinoma |

The compounds of the invention are thus useful in the treatment of leukemia and solid tumors, such as colon, colo-rectal, ovarian, mammary, prostate, lung, kidney and also melanoma tumors. The dosage range adopted will depend on the route of administration and on the age, weight and. condition of the patient being treated. The compounds may be administered, for example, by the parenteral route, for example, intramuscularly, intravenously or by bolus infusion.

As used herein, a “therapeutically effective amount” of hCPT derivatives of the present invention is intended to mean that amount of the compound which will inhibit the growth of, or retard cancer, or kill malignant cells, and cause the regression and palliation of malignant tumors, i.e., reduce the volume or size of such tumors or eliminate the tumor entirely.

With mammals, including humans, the effective amounts can be administered on the basis of body surface area. The interrelationship of dosages varies for animals of various sizes and species, and for humans (based on mg/m² of body surface) is described by E. J. Freireichet et al., Cancer Chemother. Rep., 50(4) :219 (1966). Body surface area may be approximately determined from the height and weight of an individual (see, e.g., Scientific Tables, Geigy Pharmaceuticals, Ardsley, N.Y. pp. 537-538 (1970)). A suitable dose range is from 1 to 1000 mg of equivalent per m² body surface area of a compound of the invention, for instance from 50 to 500mg/m².

For all of the administering routes, the exact timing of administration of the dosages can be varied to achieve optimal results. Generally, if using Intralipid 20 as the carrier for the hCPT derivative, the actual dosage of hCPT derivative reaching the patient will be less. This is due to some loss of the hCPT derivative on the walls of the syringes, needles and preparation vessels, which is prevalent with the Intralipid 20 suspension. When a carrier, such as cottonseed oil is used, this above described loss is not so prevalent because the hCPT derivative does not adhere as much to the surface of syringes, etc.

Another important feature of the method provided by the present invention relates to the relatively low apparent overall toxicity of the hCPT derivatives administered in accordance with the teachings herein. Overall toxicity can be judged using various criteria. For example, loss of body weight in a subject over 10% of the initially recorded body weight (i.e., before treatment) can be considered as one sign of toxicity. In addition, loss of overall mobility and activity and signs of diarrhea or cystitis in a subject can also be interpreted as evidence of toxicity.

### Process of the Invention

Another aspect of this invention is process for preparing compounds of this invention by reacting homo-camptothecin (hCPT) or a hCPT analogue with a compound of the formula R—C(O)X, wherein R is R₁—O—(CH₂) m, R₁ is as defined herein, m is an integer of 1-10, and X is e.g., bromide, chloride, hydroxy, alkoxy of 1-11 carbons (e.g., —O(CH₂)ₘ CH₃ where m is an integer of 1-10) or R—C(O)O—(R is defined hereinbefore). Preferably X is OH or lower alkoxy such as ethoxy. The compound shown as R₁—O—(CH₂)ₘ—C(O)X can be referred to as an “oxyalkanoic acid” or an “oxyalkanoic acid derivative.” One way that such an oxyalkanoic acid derivative is obtained is by reacting an appropriate alcohol (R₁OH) with an omega-halogenated alkanoic acid or by reacting an arylalcohol (e.g., a phenol or substituted phenol) with an omega-halogenated alkanoate, then hydrolyzing. Examples of such alkanoic acids include 2-bromoacetic acid, 3-bromopropanoic acid, 4-bromobutyric acid, 5-bromopentanoic acid, the corresponding alkyl esters (e.g., methyl, ethyl, propyl, and the like, preferably ethyl), the corresponding acid halides (especially the chloride), and the like. The ethyl ester of 2-bromoacetic acid is preferred. It may be useful to prepare an acid halide from the corresponding oxyalkanoic acid. The acid halides are obtained by reacting the corresponding oxyalkanoic acid with halogenated agents (such as SOCl₂, PCl₃, POCl₃, PCl₅, PBr₃, and so on). The acid chloride is preferred. Once the oxyalkanoic acid or its derivative is prepared, it is reacted with hCPT on a hCPT analogue to form the (S)-20-ester of hCPT, i.e. compounds of this invention. This reaction sequence can be generalized as follows:

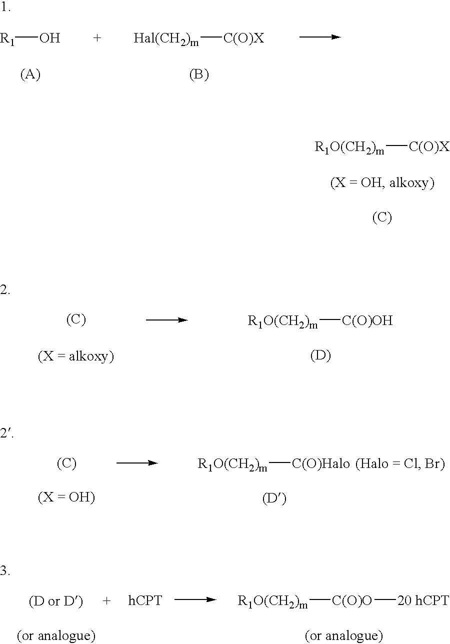

In step 1 the reaction conditions will vary depending on the exact reactants employed. In general, solvents useful in the reaction may be aqueous or nonaqueous. Preferably, a solvent will be water, an organic solvent miscible with water, or mixtures thereof. Examples of useful miscible solvents include acetone and dimethyformamide (DMF). When the solvent is aqueous, the pH of the reaction will be basic, e.g., in the range of 10 to 14, preferably about 12 to 14. The reaction temperature vary with the reactant, and the solvents, and will range from about 20° C. to about 180° C., preferably about 40° C. to about 80° C. The time needed for the reaction to be complete will generally be no more than about 10 hours, preferably about 2 to 4 hours.

In step 2, the compound of formula (C) is converted to a compound of formula (D) by a hydrolysis reaction, generally performed in two stages. The reaction conditions for this step will vary in accordance with the compound being reacted. In general, solvents useful in the conversion may be aqueous or nonaqueous, preferably, a solvent will be water, either alone or with a water-miscible organic solvent. An example of a particularly useful solvent is a mixture of water and DMF or water and dioxane. The pH of the first stage of reaction will be basic, e.g., in the range of 10 to 14, preferably about 12 to 14. A suitable inorganic base such as an alkaline earth hydroxide, e.g., sodium hydroxide, is useful. The reaction temperature will range from about 0° C. to about 60° C., preferably about 20° C. to about 25° C. The time needed for the reaction to be complete will generally be no more than 10 hours, preferably no more than about 4 hours. The mixture is then acidified to a pH of less than 4, e.g., 3, with an appropriate acid such as hydrogen chloride and extracted with a suitable solvent such as ethyl acetate in accordance with standard chemical synthetic methods.

In step 2′, the compound of formula C (i.e. the oxyalkanoic acid is converted into the corresponding acid halide by reacting with a halogenated agent such as SOCl₂, PCl₃, POCl₃, PCl₅, PBr₃, and the like under appropriate conditions.

In step 3 of the process a compound of formula (D) is reacted with hCPT or a hCPT analogue in about equimolar amounts under conditions suitable for the formation of the compounds of this invention. The reaction takes place in the presence of suitable carbodiimide compound such as disopropylcarbodiimide, but preferably 1-(3-dimethylaminopropyl)-3-ethyl carbodiimide hydrochloride (EDCI), and 4-(dimethylamino) pyridine (DMAP) in the presence of a suitable solvent, preferably a nonaqueous, nonpolar solvent. Examples of useful solvents in this step include halogenated alkanes, e.g., dichoromethane or trichloromethane) and DMF. Dichloromethane is particularly useful. The reaction temperature will range from about 20° C. to about 40° C., preferably about 20° C. to about 25° C. The time needed for the reaction to be complete will generally be no more than about 20 hours, preferably about 10 hours. It should be noted that a compound of formula (I) wherein one of R₂-R₆ is R₁—O(CH₂)ₘ—C(O)O along with R being R₁—O(CH₂)ₘ is obtained by reacting a hCPT analogue where one of R₂-R₆ (particularly R₄) is a hydroxy. In this case, the compound, e.g., the 10 hydroxy hCPT, is reacted with 2 molar amount of the oxyalkanoic acid to give the disubstituted hCPT derivative.

In step 1, suitable alcohols represented by formula (A) include the following:

butanol;

menthol;

4-nitrophenol;

sesamol;

2-bromo-4-chlorophenol;

2, 6-dichloro-4-fluorophenol;

4-nitro-2-trifluoromethylphenol;

4-cyano-3,5-dibromophenol;

6-iodo-2-picolin-5-ol;

4-(4′-acetylpiperazino)phenol;

4-bromo-3-chlorophenol;

5-bromo-2,3-difluorophenol;

4-trifluoromethoxyphenol;

2-bromo-4-fluorophenol;

4-acetyl-2-fluorophenol;

2-fluoro-5-trifluoromethylphenol;

4-hydroxyquinoline;

4-trifluoromethylphenol;

4-cyanophenol;

4-cyanophenol;

4-cyano-3,5-diiodophenol;

4-cyano-3-fluorophenol;

4-cyano-3-fluorophenol;

2-benzothiazolol; and the like.

One of skill in the art will recognize that other representative alcohols may be used provided with the guidance of this specification.

In step 2, suitable esters represented by formula (C) include the following:

ethyl 4-nitrophenoxyacetate;

ethyl 3, 4-methylenedioxyphenoxyacetate;

ethyl 2-bromo-4-chlorophenoxyacetate;

ethyl 2, 6-dichloro-4-fluorophenoxyacetate;

ethyl 4-nitro-2-trifluoromethylphenoxyacetate;

ethyl 4-cyano-3,5-dibromophenoxyacetate;

ethyl 6-iodo-2-methylpyridine-5-oxyacetate;

ethyl 4-(4′-acetylpiperazino)phenoxyacetate;

ethyl 4-bromo-3-chlorophenoxyacetate;

ethyl 5-bromo-2,3-difluorophenoxyacetate;

ethyl 4-trifluoromethoxyphenoxyacetate;

ethyl 2-bromo-4-fluorophenoxyacetate;

ethyl 4-acetyl-2-fluorophenoxyacetate;

ethyl 2-fluoro-5-trifluoromethylphenoxyacetate;

ethyl quinoline-4-oxyacetate;

ethyl 4-trifluoromethylphenoxyacetate;

ethyl 4-cyanophenoxyacetate;

ethyl 4-cyano-3,5-diiodophenoxyacetate;

ethyl 4-cyano-3-fluorophenoxyacetate;

benzothiazole-2-oxyacetate; and the like.

One of skill in the art will recognize other representative esters may be used provided the guidance of this specification.

In step 3, a suitable hCPT analogue is a compound that is hCPT substituted at the 7, 9, 10, 11, 12 or 22 positions as described in this document. The hCPT analogue may be substituted with substituents known in the art or that can be prepared by one of skill in the art given the disclosure herein (i.e., from CPT analogues). Representative articles that teach how to make CPT analogues or where such analogues may be procured are found in the following journals (which are incorporated herein by reference).

1. J. Med. Chem. 1998, 41, 31-37

2. J. Med. Chem. 2000, 43, 3970-3980

3. J. Med. Chem. 1993, 36, 2689-2700

4. J. Med. Chem. 1991, 34, 98-107

5. J. Med. Chem. 2000, 43, 3963-3969

6. Chem. Pharm. Bull. 39(10) 2574-2580 (1991)

7. Chem. Pharm. Bull. 39(6) 1446-1454 (1991)

8. ANTIMICROBIAL AGENTS AND CHEMOTHERAPY, December 1999, p.2862-2868

9. European Journal of Cancer, Vol. 34, No. 10, pp. 1500-1503, 1998

10. CANCER RESEARCH 55, 753-760, Feb. 15, 1995

11. Ant-Cancer Drug Design (1998), 13, 145-157

12. Bioorganic & Medicinal Chemistry Letters 8 (1998) 415-418

13. CANCER RESEARCH 61, 6034-6037, Aug. 15, 2001

14. Clinical Cancer Research 2002, 8, 641-661

Suitable CPT analogues include the following, where the number in parenthesis following the name refers to journal article listed above:

camptothecin (CPT);

exatecan mesylate (14-AKA DX8951f);

(20S)-7-ethyl-10-[4-(1-piperidino)-1-piperidino]carbonyloxy)-CPT (irinotecan);

(20S)-9-nitro CPT (1);

(20S)-7-chloro-n-propyldimethylsilyl CPT (2);

(20S)-10-hydroxy-7-chloro-n-propyldimethylsilyl CPT (2);

(20S)-10-acetoxy-7-chloro-n-propyldimethylsilyl CPT (2);

(20S)-7-tert-butyldimethylsilyl CPT (2);

(20S)-10-hydroxy-7-tert-butyldimethylsilyl CPT (2);

(20S)-10-acetoxy-7-tert-butyldimethylsilyl CPT (2);

(20S)-9-hydroxy CPT (3);

(20S)-9-amino CPT (3);

(20S)-10-amino CPT (3);

(20S)-9-amino-10-hydroxy CPT (3);

(20S)-9-amino-10,11-methylenedioxy CPT (3);

(20S)-9-methylamino CPT;

(20S)-9-methyl CPT (3);

(20S)-9-dimethylaminomethyl CPT;

(20S)-9-chloro CPT (3);

(20S)-9-fluoro CPT (3);

(20S)-9-trimethylsilylethyl CPT (14-AKA Karenitecin);

(20S)-7-(4-methylpiperazinomethylene)-10,11-ethylenedioxy CPT (14)

(20S)-9-piperidino CPT;

(20S)-9-dimethylaminomethyl-10-hydroxy CPT (3-AKA Topotecan);

(20S)-9-morpholinomethyl CPT (4);

(20S)-10-hydroxy CPT (3);

(20S)-9,10-dichloro CPT (3);

(20S)-10-bromo CPT (3);

(20S)-10-chloro CPT (3);

(20S)-10-methyl CPT (3);

(20S)-10-fluoro CPT (3);

(20S)-10-nitro CPT (3);

(20S)-10,11-methylenedioxy CPT (3);

(20S)-10-formyl CPT (3);

(20S)-10-nonylcarbonyloxy CPT (12);

(20S)-10-undecylcarbonyloxy CPT (12);

(20S)-10-pentadecylcarbonyloxy CPT (12);

(20S)-10-heptadecylcarbonyloxy CPT (12);

(20S)-10-nonadecylcarbonyloxy CPT (12);

(20S)-9-nitro-10,11-methylenedioxy CPT (3);

(20S)-9-(4-methylpiperazinylmethyl)-10-hydroxy (CPT) (4);

(20S)-9-[4-(1-piperidino)-1-piperidinomethyl]-10-hydroxy CPT (4);

(20S)-9-methyl-10,11-methylenedioxy CPT;

(20S)-9-chloro-10,11-methylenedioxy CPT (3);

(20S)-9-cyano-10,11-methylenedioxy CPT;

(20S)-9-acetoxy-10,11-methylenedioxy CPT;

(20S)-9-acetylamino-10,11-methylenedioxy CPT;

(20S)-9-aminomethyl-10-hydroxy CPT;

(20S)-9-ethoxymethyl-10-hydroxy CPT (4);

(20S)-9-methylaminomethyl-10-hydroxy CPT;

(20S)-9-n-propylaminomethyl-10-hydroxy CPT (4);

(20S)-9-dimethylaminomethyl-10-hydroxy CPT (4);

(20S)-9-cyclohexylaminomethyl-10-hydroxy CPT (4);

(20S)-9-(2-hydroxyethyl) aminomethyl-10-hydroky CPT (4);

(20S)-9-(trimethylammonio)methyl-10-hydroxy CPT, methanesulfonate (4);

(20S)-9-morpholinomethyl-10-hydroxy CPT (4);

(20S)-9-cyanomethyl-10-hydroxy CPT (4);

(20S)-CPT-7-aldehyde (5);

(20S)-10-methoxy CPT-7-aldehyde (5);

(20S)-7-acetoxymethyl CPT (5);

(20S)-7-acetoxymethyl-10-methyl CPT (5);

(20S)-7-cyano-10-methoxy CPT (5);

(20S)-7-cyano CPT (5);

(20S)-7-formylethenyl CPT (5);

(20S)-7-ethoxycarbonylethenyl CPT (5);

(20S)-7-cyanoethenyl CPT (5);

(20S)-7-(2,2-dicyanoethenyl) CPT (5);

(20S)-7-(2-cyano-2-ethoxycarbonyl) ethenyl CPT (5);

(20S)-7-ethoxycarbonylethyl CPT (5);

(20S)-7-ethyl CPT (6);

(20S)-7-n-propyl CPT (6);

(20S)-7-acetoxymethyl CPT (6);

(20S)-7-n-propylcarbonyloxymethyl CPT (6);

(20S)-7-ethoxycarbonyl CPT (6);

(20S)-7-ethyl-10-hydroxy CPT;

(20S)-7-ethyl-10-acetyloxy CPT;

(20S)-7-methyl-10-aminocarbonyloxy CPT;

(20S)-7-n-propyl-10-piperidinocarbonyloxy CPT;

(20S)-7-ethyl-10-(2-dimethylamino) ethyl CPT;

(20S)-7-ethyl-10-carbamoyloxy derivatives of CPT;

(20S)-7-ethyl-10-[4-(1-piperidino)-piperidino carbonyloxy CPT (7);

(20S)-7-ethyl-10-(1-piperazine) carbonyloxy CPT (7);

(20S)-7-ethyl-10-(4-i-propylaminocarbonylmethylpiperazine) carbonyloxy CPT (7);

(20S)-7-ethyl-10-[4(1-pyrrolidinyl) piperazine]carbonyloxy CPT (7);

(20S)-7-ethyl-10-[(4-(dimethylamino)-1-piperidino]carbonyloxy CPT (7);

(20S)-7-ethyl-10-[4-(di-n-propylamino)-1-piperidinol]carbonyloxy CPT (7);

(20S)-7-ethyl-10-[(4-(di-n-butylamino)-1-piperidino]carbonyloxy CPT (7);

(20S)-7-ethyl-10-[4-(1-pyrrolidino)-1-piperidino)]carbonyloxy CPT (7);

(20S)-7-butoxyiminomethyl CPT;

(20S)-7-ethyl-10-[4-(1-piperidino)-1-piperidino]carbonyloxy CPT (7); and

(20S)-7-ethyl-10-[N-methyl-N-2-(dimethylamino)ethylamino]carbonyloxy CPT (7) and the like.

CPT and CPT analogues may be converted to hCPT and hCPT analogues by the following partial synthesis illustrated for CPT (Lavergne et al., Ann. N.Y. Acad. Sci. 2000, 922:100-111).

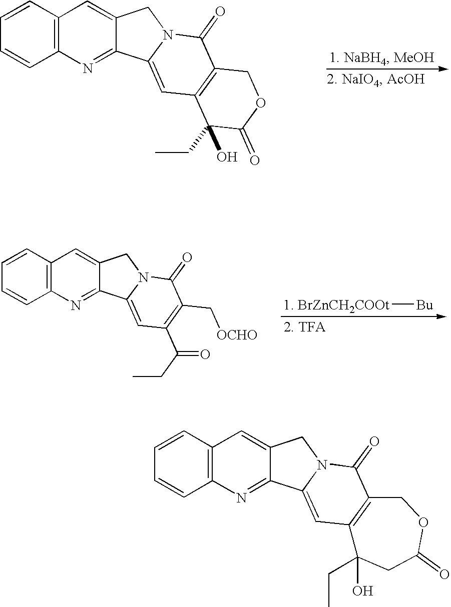

Alternatively, total synthesis of hCPT analogues may be performed by analogy to the procedure described in Lavergne et al., Ann. N.Y. Acad. Sci. 2000, 922: 100-111.

It will be recognized by one of skill in the art that other similar compounds may be prepared by following the teachings set forth in the above articles and modifying with appropriate art-recognized steps.

In step 3, suitable oxyalkanoic acids of formula (D) including the following: peony acetic acid;

4-fluorophenoxyacetic acid;

4-bromophenoxyacetic acid;

4-iodophenoxyacetic acid;

4-chlorophenoxyacetic acid;

2,3-dichlorophenoxyacetic acid;

4-methoxyphenoxyacetic acid;

2-nitrophenoxyacetic acid;

4-nitro-3-trifluoromethylphenoxyacetic acid;

4-cyano-3-fluorophenoxyacetic acid;

4-methylphenoxyacetic acid;

4-chloro-2-methylphenoxyacetic acid;

3-bromomethylphenoxyacetic acid;

4-benzyloxyphenoxyacetic acid;

4-isopropylphenoxyacetic acid;

4-formylphenoxyacetic acid;

2,3,4,5,6-pentafluorophenoxyacetic acid;

4-carboxyphenoxyacetic acid;

2,6-dichloro-4-fluorophenoxyacetic acid;

3,4-methylenedioxyphenoxyacetic acid;

6-iodo-2-methylpyridine-5-oxyacetic acid;

quinoline-4-oxyacetic acid;

(−)-menthoxyacetic acid;

7-(carboxymethoxy)-3-chloro-4-methylcoumarin;

7-(carboxymethoxy)-4-methylcoumarin;

2-naphthoxyacetic acid;

benzothiazole-2-oxyacetic acid;

ethyloxyacetic acid;

butyloxyacetic acid;

cyclohexyloxyacetic acid;

phenoxypropanoic acid;

phenoxybutyric acid;

4-acetyl-2-fluorophenoxyacetic acid;

4-(4′-acetylpiperazino) phenoxyacetic acid;

2-bromo-4-chlorophenoxyacetic acid;

2-bromo-4-fluorophenoxyacetic acid;

2-bromo-4-fluorophenoxyvaleric acid;

4-bromo-3-chlorophenoxyacetic acid;

2-chlorophenoxyacetic acid;

3-chlorophenoxyacetic acid;

4-chloro-3,5-dimethylphenoxyacetic acid;

4-chloro-2-methylphenoxybutyric acid;

4-cyanophenoxyacetic acid;

4-cyano-2,6-dibromophenoxyacetic acid;

4-cyano-2,6-diiodophenoxyacetic acid;

3,5-ditrifluoromethylphenoxyacetic acid;

2,3-difluoro-5-bromophenoxyacetic acid;

2,4-dichlorophenoxyacetic acid;

2,4-dichlorophenoxybutyric acid;

2,4-dimethylphenoxyacetic acid;

4-ethylphenoxyacetic acid;

2-i.propyl-5-methylphenoxyacetic acid;

2-fluoro-5-rifluoromethylphenoxyacetic acid;

3-methoxyphenoxyacetic acid;

4-methoxyphenoxyacetic acid;

4-nitrophenoxyacetic acid;

4-trifluoromethoxyphenoxyacetic acid;

4-trifluoromethylphenoxyacetic acid; and the like.

One of skill in the art will recognize that other similar oxyalkanoic acids may be obtained from commercial sources or prepared by art-recognized procedures to be used in step 3 to prepare compounds of this invention. By reacting a compound shown in the list of hCPT analogues with a compound shown in the list of compounds of formula (D) in accordance with the guidelines for reaction condition, compounds of the invention will be obtained. These compounds will exhibit the desired characteristics to a greater or lesser extent. Guidance is provided herein as to the preferred subgroups of compounds within the family.

### EXAMPLES

The following examples provide representative compounds included as part of the current invention. The examples also provide descriptions of in vitro and in vivo assays to aid in determining the utility of the compounds. The homo-camptothecin esters in examples 1-28 are prepared from the corresponding oxyacetic acid and homo-camptothecin. Throughout the examples chemical formulas are used to name compounds (e.g., NaHCO₃ is sodium bicarbonate) as appropriate.

All reactions were carried out in oven-dried glassware cooled under vacuum. Flash column chromatography was performed on EM Science silica gel (230-400 mesh). ¹H NMR spectra were obtained at ambient temperature on a Varian 600 MHz instrument with chemical shifts reported relative to TMS as an internal standard. Melting points were recorded on a Melt-temp II, Laboratory Devices, Inc. and are uncorrected. Anhydrous THF, dichloromethane, ether and chloroform were dried with molecular sieves prior to use. All chemical yields reported refer to a single run of a compound estimated to be greater than 95% pure as determined by ¹H NMR. Racemic homocamptothecin was synthesized as previously reported in the literature. (Lavergne, O.; Lesueur-Ginot, L.; Rodas, F. P.; Kasprzyk, P. G.; Pommier, J.; Demarquay, D.; Prevost, G.; Ulibarri, G.; Rolland, A.; Schiano-Liberatore, A.-M.; Harnett, J.; Pons, D.; Camara, J.; Bigg, D. C. H. J. Med. Chem. 1998, 41, 5410.) All reagents are commercially available and were used without further purification. The procedures described in the following sections are representative.

Representative procedure for the synthesis of homocamptothecin-20-O phenoxyacetic acid esters: An oven-dried round bottom flask containing a magnetic stirbar was allowed to cool to room temperature under vacuum and was then backfilled with nitrogen. The flask was charged with homocamptothecin (hCPT) (20 mg, 0.05 mmol) and then 5 mL of dry chloroform. The solution was allowed to stir for 10 minutes at ambient temperature. 1-(3-Dimethylamino-propyl)-3-ethyl-carbodiimide hydrochloride (EDCI) (44 mg, 0.23 mmol), 4-(dimethylamino)pyridine (DMAP) (5 mg, 0.036 mmol) and the appropriate oxyacetic acid (30 mg, 0.14 mmol) were added. The reaction was allowed to stir for about 20 hours at ambient temperature. The reaction mixture was diluted with dichloromethane (25 mL) and washed with saturated aqueous sodium bicarbonate solution (3×15 mL). The organic layer was dried over anhydrous magnesium sulfate, filtered and concentrated under reduced pressure. The crude product was purified by flash chromatography (silica gel, 25:75 hexanes-ethyl acetate) to afford, after concentration of the appropriate fractions and removal of traces of solvent (vacuum pump), homocamptothecin-20-ester of the appropriate oxyacetic acid as a solid. Spectral data and a melting point were obtained.

### Example 1

This example explains how to prepare non-substituted and substituted homo-camptothecin-20-O-esters of 4-fluorophenoxyacetic acid.

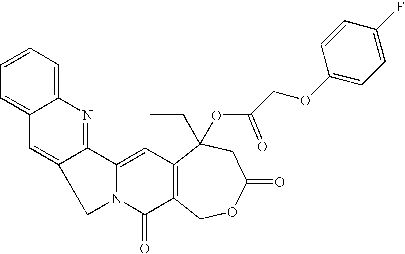

A. Homo-camptothecin-20-O-ester of 4-Fluorophenoxyacetic Acid (1160)

The target molecule was synthesized using the procedure for the synthesis of homocamptothecin-20-O phenoxyacetic acid esters. The flask was charged with homocamptothecin (hCPT) (30 mg, 0.08 mmol) and then 10 mL of dry chloroform. The solution was allowed to stir for 10 minutes at ambient temperature. 1-(3-Dimethylaminopropyl)-3-ethyl-carbodiimide hydrochloride (EDCI) (66 mg, 0.34 mmol), 4-(dimethylamino)pyridine (DMAP) (7 mg, 0.058 mmol) and 4-fluorophenoxyacetic acid (42 mg, 0.25 mmol) were added. The reaction was allowed to stir for 16 hours at ambient temperature. The reaction was quenched and washed as described in the general procedure. The crude product was purified by flash chromatography (silica gel, 75:25 ethyl acetate-hexanes) to afford, after concentration of the appropriate fractions and removal of traces of solvent (vacuum pump), 0.015 g, 35% of the title compound as an yellow solid. m.p.: 142-144° C. ¹H NMR (400 MHz, CDCl₃) δ 8.4 (s, 1H), 8.2 (d, 1H), 7.9 (d, 1H), 7.85 (t, 1H), 7.70 (t, 1H), 7.20 (s, 1H), 7.95 (m, 2H), 6.85 (m, 2H), 5.85(d, 1H), 5.5 (d, 1H), 5.3 (s, 2H), 4.7 (dd, 2H), 4.4 (d, 1H), 3.1 (d, 1H), 2.3 (m, 11H), 1.9 (m, 2H), 1.3 (s, 2H), 1.1 (s, 3H).

B. By substituting other homo-camptothecin analogues for homo-camptothecin (hCPT) in part A of this example other compounds of this invention are prepared. In naming homo-camptothecin analogues, the standard numbering system for homo-camnptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 2

This example explains how to prepare non-substituted and substituted homo-camptothecin-20-O-esters of 4-trifluoromethoxyphenoxyacetic acid.

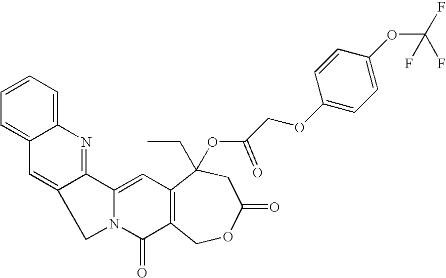

A. Homo-camptothecin-20-O-ester of 4-Trifluoromethoxyphenoxyacetic Acid (1164)

The target molecule was synthesized using the procedure for the synthesis of homocamptothecin-20-O phenoxyacetic acid esters. The flask was charged with homocamptothecin (hCPT) (20 mg, 0.05 mmol) and then 5 mL of dry chloroform. The solution was allowed to stir for 10 minutes at ambient temperature. 1-(3-Dimethylaminopropyl)-3-ethyl-carbodiimide hydrochloride (EDCI) (44 mg, 0.23 mmol), 4-(dimethylamino)pyridine (DMAP) (5 mg, 0.036 mmol) and 4-trifluoromethoxyphenylacetic acid (30 mg, 0.14 mmol) were added. The reaction was allowed to stir for 16 hours at ambient temperature. The reaction was quenched and washed as described in the general procedure. The crude product was purified by flash chromatography (silica gel, 75:25 ethyl acetate-hexanes) to afford, after concentration of the appropriate fractions and removal of traces of solvent (vacuum pump), 0.004 g, 17% of the title compound as a pale yellow solid. m.p.: 204-206° C. ¹H NMR (600 MHz, DMSO-d₆) δ 8.6 (s, 1H), 8.15 (d, 1H, J=8 Hz), 8.05 (d, 1H, J=8 Hz), 7.8 (m, 1H), 7.65 (m, 1H), 7.16 (s, 1H), 6.95 (m, 4H), 5.5 (d, 1H, J=15 Hz), 5.4 (d, 1H, J=15 Hz), 5.2 (s, 2H), 4.95 (d, 1H, J=17 Hz), 4.85 (d, 1H, J=17 Hz), 3.6 (d, 1H, J=14 Hz), 3.4 (d, 1H, J=14 Hz), 2.3 (m, 1H), 1.8 (m, 1H), 1.3(m, 2H) 1.1 (s, 3H).

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 3

This example explains how to prepare non-substituted and substituted homo camptothecin-20-O-esters of 2,3-dichlorophenoxyacetic acid.

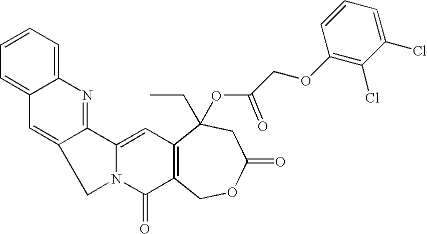

A. Homocamptothecin-20-O Ester of 2,3-Dichlorophenoxy Acetic Acid (1158)

The target molecule was synthesized using the procedure for the synthesis of homocamptothecin-20-O phenoxyacetic acid esters. The flask was charged with homocamptothecin (hCPT) (20 mg, 0.05 mmol) and then 5 mL of dry chloroform. The solution was allowed to stir for 10 minutes at ambient temperature. 1-(3-Dimethylaminopropyl)-3-ethyl-carbodiimide hydrochloride (EDCI) (44 mg, 0.23 mmol), 4-(dimethylamino)pyridine (DMAP) (5 mg, 0.036 mmol) and 2,3-dichlorophenoxyacetic acid (32 mg, 0.14 mmol) were added. The reaction was allowed to stir for 16 hours at ambient temperature. The reaction was quenched and washed as described in the general procedure. The crude product was purified by flash chromatography (silica gel, 75:25 ethyl acetate-hexanes) to afford, after concentration of the appropriate fractions and removal of traces of solvent (vacuum pump), 0.005 g, 18% of the title compound as a yellow solid. m.p.: 182-185° C. ¹H NMR (600 MHz, CDCl₃) δ 8.4 (s, 1H), 8.2 (d, 1H), 7.9 (d, 1H), 7.85 (t, 1H), 7.70 (t, 1H), 7.20 (s, 1H), 7.0 (s, 1H), 6.7 (m, 2H), 5.9 (d, 1H), 5.5 (d, 1H), 5.3 (s, 2H), 4.4 (d, 1H), 3.1 (d, 1H), 2.3 (m, 1H), 1.9 (m, 1H), 1.25 (m, 2H), 1.1 (s, 3H).

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 4

This example explains how to prepare non-substituted and substituted homocamptothecin-20-O-esters of 2-chlorophenoxyacetic acid.

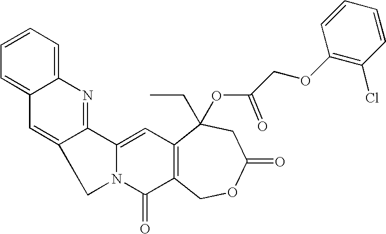

A. Homocamptothecin-20-O Ester of 2-Chlorophenoxy Acetic Acid (021229)

The target molecule was synthesized using the procedure for the synthesis of homocamptothecin-20-O phenoxyacetic acid esters. The flask was charged with homocamptothecin (hCPT) (27 mg, 0.07 mmol) and then 10 mL of dry chloroform. The solution was allowed to stir for 10 minutes at ambient temperature. 1-(3-Dimethylamino-propyl)-3-ethyl-carbodiimide hydrochloride (EDCI) (75 mg, 0.39 mmol), 4-(dimethylamino)pyridine (DMAP) (6 mg, 0.043 mmol) and 2,3-dichlorophenoxyacetic acid (35 mg, 0.19 mmol) were added. The reaction was allowed to stir for 20 hours at ambient temperature. The reaction was quenched and washed as described in the general procedure. The crude product was purified by flash chromatography (silica gel, 75:25 ethyl acetate-hexanes) to afford, after concentration of the appropriate fractions and removal of traces of solvent (vacuum pump), 0.008 g, 20% of the title compound as a yellow solid. ¹H NMR (400 MHz, CDCl₃) δ 8.4 (s, 1H), 8.25 (d, 1H), 7.9 (d, 1H), 7.8 (t, 1H), 7.7 (t, 1H), 7.35 (dd, 1H), 7.20 (s, 1H), 7.10 (s, 1H), 6.9 (m, 2H), 5.85 (d, 1H), 5.45 (d, 1H), 5.3 (s, 2H), 4.4 (d, 1H), 3.05 (d, 1H), 2.3 (m, 1H), 1.9 (m, 1H), 1.25 (m, 2H), 1.1 (s, 3H).

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation -for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 5

This example explains how to prepare non-substituted and substituted homo-camptothecin-20-70-esters of 4-methoxyphenoxyacetic acid.

A. Homo-camptothecin-20-O-ester of 4-Methoxyphenoxyacetic Acid

A mixture of homo-camptothecin (10 mg, 0.029 mmol), 4-methoxyphenoxyacetic acid (11 mg, 0.060 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-4-methoxyphenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7 ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 6

This example explains how to prepare non-substituted and substituted homo-camptothecin-20-O-esters of 2-nitrophenoxyacetic acid.

A. Homo-camptothecin-20-O-ester of 2-Nitrophenoxyacetic Acid

A mixture of homo-camptothecin (10 mg, 0.029 mmol), 2-nitrophenoxyacetic acid (8.2 mg, 0.042 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-4-methoxyphenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9 methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 7

This example explains how to prepare non-substituted and substituted homocamptothecin-20-O-esters of 4-methylphenoxyacetic acid.

A. Homo-camptothecin-20-O-ester of 4-Methylphenoxyacetic Acid

A mixture of homo-camptothecin (10 mg, 0.029 mmol), 4-methylphenoxyacetic acid (10 mg, 0.63 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-4-methylphenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7 ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 8

This example explains how to prepare non-substituted and substituted homo-camptothecin-20-O-esters of 4-chloro-2-methylphenoxyacetic acid.

A. Homo-camptothecin-20-O-ester of 4-Chloro-2-methylphenoxyacetic Acid

A mixture of homo-camptothecin (30 mg, 0.086 mmol), 4-chloro-2-methylphenoxyacetic acid (30 mg, 0.15 mmol), EDCI (50 mg, 0.26 mmol), DMAP (5 mg, 0.05 mmol) and dichloromethane (4 ml) is stirred at room temperature for 20 h, then dichloromethane (20 ml) is added to the solution. The organic layer is washed with water (20 ml), saturated NaHCO₃ aqueous solution (20 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-4-chloro-2-methyl yield.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino HCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10, 11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-i10-(2-dimethylamino) ethyl hCPT; and the like.

### Example 9

This example explains how to prepare non-substituted and substituted homo-camptothecin-20-O-esters of 7-(carboxymethoxy)-3-chloro-4-methylcoumarin.

A. Homo-camptothecin-20-O-ester of 7-(Carboxymethoxy)-3-chloro-4-methylcoumarin

The mixture of homo-camptothecin (10 mg, 0.029 mmol), 7-(carboxymethoxy)-3-chloro-4-methylcoumarin (11 mg, 0.042 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-ester of 7-(carboxymethoxy)-3-chloro-4-methylcoumarin.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10, 11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,1-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl)aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 10

This example explains how to prepare non-substituted and substituted homo-camptothecin-20-O-esters of 2-naphthoxyacetic acid.

A. Homo-camptothecin-20-O-ester of 2-Naphthoxyacetic Acid

The mixture of homo-camptothecin (10 mg, 0.029 mmol), 2-naphthoxyacetic acid (8.5 mg, 0.042 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-2-naphthoxyacetate.

B. By substituting other homo-camptothecin analogues for homo-camptothecin (hCPT) in part A of this example other compounds of this invention are prepared. In naming homo-camptothecin analogues;) the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 11

This example explains how to prepare non-substituted and substituted homo-camptothecin-20-O-esters of 3-bromomethylphenoxyacetic acid.

A. Homo-camptothecin-20-O-ester of 3-Bromomethylphenoxyacetic Acid

The mixture of homo-camptothecin (10 mg, 0.029 mmol), 3-bromomethylphenoxyacetic acid (15 mg, 0.63 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-3-bromomethylphenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,1-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 12

This example explains how to prepare non-substituted and substituted homo-camptothecin-20-O-esters of 4-benzyloxyphenoxyacetic acid.

A. Homo-camptothecin-20-O-ester of 4-Benzyloxyphenoxyacetic Acid

The mixture of homo-camptothecin (10 mg, 0.029 mmol), 4-benzyloxyphenoxyacetic acid (16 mg, 0.063 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-4benzyloxyphenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 13

This example explains how to prepare non-substituted and substituted homo-camptothecin-20-O-esters of 4-isopropylphenoxyacetic acid.

A. Homo-camptothecin-20-O-ester of 4-Isopropylphenoxyacetic Acid

The mixture of homo-camptothecin (10 mg, 0.029 mmol), 4-isopropylphenoxyacetic acid (8 mg, 0.42 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO4. After the solvent is removed under reduced pressure, the resulting solid is separated by column chromatography (eluent: CHCl₃: CH₃OH 9:1) to afford homo-camptothecin-20-O-4-isopropylphenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10, 11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 14

This example explains how to prepare non-substituted, and substituted homo-camptothecin-20-O-estets of 4-formylphenoxyacetic acid.

A. Homo-camptothecin-20-O-ester of 4-Formylphenoxyacetic Acid

The mixture of homo-camptothecin (10 mg, 0.029 mmol), 4-formylphenoxyacetic acid (7.6 mg, 0.42 mmol), EDCI (28 mg, 0:15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9: 1) to afford homo-camptothecin-20-O-4-formylphenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 15

This example explains how to prepare non-substituted and substituted homo-camptothecin-20-O-esters of 2,3,4,5,6-pentafluorophenoxyacetic acid.

A. Homo-camptothecin-20-O-ester of 2,3,4,5,6-Pentafluorophenoxyacetic Acid

The mixture of homo-camptothecin (10 mg, 0.029 mmol), 2,3,4,5,6-pentafluorophenoxyacetic acid (10 mg, 0.042 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃₀H 9:1) to afford homo-camptothecin-20-O-2,3,4,5,6-pentafluorophenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7 ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 1,6

This example explains how to prepare non-substituted and substituted homo-camptothecin-20-O-esters of 4-carboxyphenoxyacetic acid.

A. Homo-camptothecin-20-O-ester of 4-Carboxyphenoxyacetic Acid

The mixture of homo-camptothecin (30 mg, 0.086 mmol), 4-carboxyphenoxyacetic acid (40 mg, 0.20 mmol), EDCI (65 mg, 0.34 mmol), DMAP (2 mg, 0.02 mmol), dichloromethane (2 ml) and DMF (2 ml) is stirred at room temperature for 20 h, then dichloromethane (20 ml) is added to the solution. The organic layer is washed with water (20 ml) and brine (20 ml), and then dried over MgSO₄. After the solvent is removed under reduced pressure, the resulting solid is separated by column chromatography (eluent: CHCl₃: CH₃OH 7:3) to afford homo-camptothecin-20-O-4-carboxyphenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 17

This example explains how to prepare non-substituted and substituted homo-camptothecin-20-O-esters of ethyloxyacetic acid.

A. Homo-camptothecin-20-O-ester of Ethyloxyacetic Acid

The mixture of homo-camptothecin (10 mg, 0.029 mmol), ethyloxyacetic acid (10 mg, 0.10 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-ethyloxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT 7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 18

This example explains how to prepare non-substituted and substituted homo-camptothecin-20-O-esters of butyloxyacetic acid.

A. Homo-camptothecin-20-O-ester of Butyloxyacetic Acid

The mixture of homo-camptothecin (10 mg, 0.029 mmol), butyloxyacetic acid (15.5 mg, 0.12 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-butyloxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 19

This example explains how to prepare non-substituted and substituted homo-camptothecin-20-O-esters of (−)-menthoxyacetic acid.

A. Homo-camptothecin-20-O-ester of (−)-Menthoxyacetic Acid

The mixture of homo-camptothecin (10 mg, 0.029 mmol), (−)-menthoxyacetic acid (10.3 mg, 0.048 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-(−)-menthoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 20

The homo-camptothecin esters in examples 21-28 were prepared by first preparing the oxyacetic acid and then conducting the ester reaction.

This example explains how to prepare non-substituted and substituted homo-camptothecin-20-O-esters of 2,6-dichloro-4-fluorophenoxyacetic acid.

A. Homo-camptothecin-20-O-ester of 2,6-Dichloro-4-fluorophenoxyacetic Acid

Synthesis of 2,6-dichloro-4-fluorophenoxyacetic acid

The mixture of 2,6-dichloro-4-fluorophenol (362 mg, 2.0 mmol), potassium carbonate (910 mg, 6.6 mmol), ethyl bromoacetate (500 mg, 3.0 mmol) and acetone (25 ml) was refluxed for 12 h. After cooling, the mixture was filtered to remove potassium carbonate. The filtrate was concentrated under reduced pressure. To this residue, 10 ml dioxane and 14 ml 5% sodium hydroxide solution were added. After the mixture was stirred at room temperature overnight, it was acidified with concentrated hydrochloric acid to pH 2, and then extracted three times with ethyl acetate (15 ml each). The organic phases were combined, washed with water and brine, dried over magnesium sulfate, filtered, and then evaporated in vacuo. The residue was recrystallized from ethyl acetate and petroleum ether to give 348 mg 2,6-dichloro-4-fluorophenoxyacetic acid as white crystals, mp 155-158° C., yield: 72.8%.

Synthesis of homo-camptothecin-20-O-ester of 2,6-dichloro-4-fluorophenoxyacetic acid

The mixture of homo-camptothecin (10 mg, 0.029 mmol), 2,6-dichloro-4fluorophenoxyacetic acid (13 mg, 0.058 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-2,6-dichloro-4-fluorohohenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 21

This example explains how to prepare non-substituted and substituted homo-camptothecin-20-O-esters of 3,4-methylenedioxyphenoxyacetic acid.

A. Homo-camptothecin-20-O-ester of 3,4-Methylenedioxyphenoxyacetic Acid

Synthesis of, 3,4-Methylenedioxyphenoxyacetic Acid

The mixture of sesamol (270 mg, 2.0 mmol), potassium carbonate (910 mg, 6.6 mmol), ethyl bromoacetate (2.55 ml, 22.9 mmol) and acetone (25 ml) was refluxed for 21 h. After cooling, the mixture was filtered to remove potassium carbonate. The filtrate was concentrated under reduced pressure. To this residue, 20 ml dioxane and 14 ml 5% sodium hydroxide solution were added. After the mixture was stirred at room temperature overnight, it was acidified with concentrated hydrochloric acid to pH 2, and then extracted three times with ethyl acetate (20 ml each). The organic phases were combined, washed with water and brine, dried over magnesium sulfate, filtered, and then evaporated in vacuo. The residue was recrystallized from chloroform and acetone to give 45 mg 3,4-methylenedioxyphenoxyacetic acid as white crystals, mp 149-151° C.

Synthesis of homo-camptothecin-20-O-ester of 3,4-methylenedioxyphenoxyacetic acid

The mixture of homo-camptothecin (10 mg, 0.029 mmol), 3,4-methylenedioxyphenoxyacetic acid (8 mg, 0.042 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-3,4-methylenedioxy

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 22

This example explains how to prepare non-substituted and substituted homo-camptothecin-20-O-esters of 6-iodo-2-methylpyridine-5-oxyacetic acid.

A. Homo-camptothecin-20-O-ester of 6-Iodo-2-methylpyridine-5-oxyacetic Acid

Synthesis of 6-iodo-2-methylpyridine-5-oxyacetic acid

The mixture of 6-iodo-2-picolin-5-ol (235 mg, 1.0 mmol), potassium carbonate (455 mg, 3.3 mmol), ethyl bromoacetate (250 mg, 1.49 mmol) and acetone (15 ml) was refluxed for 15 h. After cooling, the mixture was filtered to remove potassium carbonate. The filtrate was concentrated under reduced pressure. To this residue, 10 ml of dioxane and 14 ml 5% sodium hydroxide solution were added. After the mixture was stirred at room temperature overnight, it was acidified with concentrated hydrochloric acid to pH 3, and then extracted three times with ethyl acetate (20 ml each). The organic phases were combined, washed with water and brine, dried over magnesium sulfate, filtered, and then evaporated in vacuo. The residue was recrystallized from ethanol and petroleum ether to give 165 mg 6-iodo-2′methylpyridine-5-oxyacetic acid as white crystals, mp 170-172° C.

Synthesis of homo-camptothecin-20-O-ester of 6-iodo-2-methylpyridine-5-oxyacetic acid

The mixture of homo-camptothecin (10 mg, 0.029 mmol), 6-iodo-2-methylpyridine-5-oxyacetic acid (18 mg, 0.063 mmol), EDCI (28 mg, 0.15 mmol), DMAP (2 mg, 0.02 mmol) and dichloromethane (3 ml) was stirred at room temperature for 20 h, then dichloromethane (20 ml) was added to the solution. The organic layer was washed with water (20 ml), saturated NaHCO₃ aqueous solution (10 ml) and brine (20 ml), and then dried over MgSO₄. After the solvent was removed under reduced pressure, the resulting solid was separated by column chromatography (eluent: CHCl₃: CH₃OH 9:1) to afford homo-camptothecin-20-O-6-iodo-2-methyl

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 23

This example explains how to prepare non-substituted and substituted homo-camptothecin-20-O-esters of benzothiazole-2-oxyacetic acid.

A. Homo-camptothecin-20-O-ester of Benzothiazole-2-oxyacetic Acid

Synthesis of benzothiazole-2-oxyacetic acid

The mixture of 2-benzothiazolol (320 mg, 2.1 mmol), potassium carbonate (910 mg, 6.6 mmol), ethyl bromoacetate (500 mg, 3.0 mmol) and acetone (25 ml) was refluxed for 15 h. After cooling, the mixture was filtered to remove potassium carbonate. The filtrate was concentrated under reduced pressure. To this residue, 10 ml of dioxane and 14 ml 5% sodium hydroxide solution were added. After the mixture was stirred at room temperature overnight, it was acidified with hydrochloric acid to pH 1, and then extracted three times with ethyl acetate (20 ml each). The organic phases were combined, washed with water and brine, dried over magnesium sulfate, filtered, and then evaporated in vacuo. The residue was recrystallized from ethanol and petroleum ether to give 290 mg benzothiazole-2-oxyacetic acid as white crystals, mp 168-170° C.

Synthesis of homo-camptothecin-20-O-ester of benzothiazole-2-oxyacetic acid

The mixture of homo-camptothecin (10 mg, 0.029 mmol), benzothiazole-2-oxyacetic acid (18 mg, 0.063 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-benzothiazole-2-oxyacetate.

B. By substituting other homo-camptothecin analogues for homo-camptothecin (hCPT) in part A of this example other compounds of this invention are prepared. In naming homo-camptothecin analogues, the -standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 24

This example explains how to prepare non-substituted and substituted homo-camptothecin-20-O-esters of 4-cyano-3-fluorophenoxyacetic acid.

A. Homo-camptothecin-20-O-ester of 4-Cyano-3-fluorophenoxyacetic Acid

Synthesis of 4-cyano-3-fluorophenoxyacetic acid

The mixture of 2-fluoro-4-hydroxybenzonitrile (274 mg, 2.1 mmol), potassium carbonate (910 mg, 6.6 mmol), ethyl bromoacetate (500 mg, 3.0 mmol) and acetone (25 ml) was refluxed for 6 h. After cooling, the mixture was filtered to remove potassium carbonate. The filtrate was concentrated under reduced pressure. To this residue, 10 ml of dioxane and 14 ml 5% sodium hydroxide solution were added. After the mixture was stirred at room temperature overnight, it was acidified with concentrated hydrochloric acid to pH 1, and then extracted three times with ethyl acetate (20 ml each). The organic phases were combined, washed with water and brine, dried over magnesium sulfate, filtered, and then evaporated in vacuo. The residue was recrystallized from ethanol and petroleum ether to give 268 mg 4-cyano-3-fluorophenoxyacetic acid as white crystals, mp 150-152° C.

Synthesis of homo-camptothecin-20-O-ester of 4-cyano-3-fluorophenoxyacetic acid

The mixture of homo-camptothecin (10 mg, 0.029 mmol), 4-cyano-3-fluorophenoxyacetic acid (12 mg, 0.062 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O4-cyano-3-fluorophenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino 10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9.-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 25

This example explains how to prepare non-substituted and substituted homo-camptothecin-20-O-esters of quinoline-4-oxyacetic acid.

A. Homo-camptothecin-20-O-ester of Quinoline-4-oxyacetic Acid

Synthesis of quinoline-4-oxyacetic acid

The mixture of 4-hydroxyquinoline (290 mg, 2.0 mmol), potassium carbonate (910 mg, 6.6 mmol), ethyl bromoacetate (500 mg, 3.0 mmol) and acetone (25 ml) was refluxed for 8 h. After cooling, the mixture was filtered to remove potassium carbonate. The filtrate was concentrated under reduced pressure. To this residue, 10 ml of dioxane and 14 ml 5% sodium hydroxide solution were added. After the mixture was stirred at room temperature overnight, it was acidified with concentrated hydrochloric acid to pH 3, and then extracted three times with ethyl acetate (20 ml each). The water layer was placed in a cold room, and solid came out. The crystals were filtered with suction and washed with water, and then dried to give 120 mg quinoline-4-oxyacetic acid as gray crystals, mp 274-276° C.

Synthesis of homo-camptothecin-20-O-ester of quinoline-4-oxyacetic acid

The mixture of homo-camptothecin (10 mg, 0.029 mmol), quinoline-4-oxyacetic acid (12.8 mg, 0.063 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-quinoline-4-oxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 26

This example explains how to prepare non-substituted and substituted homo-camptothecin-20-O-esters of 4-nitro-3-trifluoromethylphenoxyacetic acid.

A. Homo-camptothecin-20-O-ester of 4-Nitro-3-trifluoromethylphenoxyacetic Acid

Synthesis of 4-nitro-3-trifluoromethylphenoxyacetic acid

The mixture of 4-nitro-3-trifluoromethylphenol (414 mg, 2.0 mmol), potassium carbonate (910 mg, 6.6 mmol), ethyl bromoacetate (500 mg, 3.0 mmol) and acetone (25 ml) was refluxed for 8 h. After cooling, the mixture was filtered to remove potassium carbonate. The filtrate was concentrated under reduced pressure. To this residue, 10 ml of dioxane and 14 ml 5% sodium hydroxide solution were added. After the mixture was stirred at room temperature overnight, it was acidified with concentrated hydrochloric acid to pH 2, and then extracted three times with ethyl acetate (20 ml each). The organic phases were combined, washed with water and brine, dried over magnesium sulfate, filtered, and then evaporated in vacuo. The residue was separated by column chromatography (eluent: ethyl acetate: EtOH 9:1) to give 35 mg 4-nitro-3-trifluoromethylphenoxyacetic acid as white solid, mp 92-95° C.

Synthesis of homo-camptothecin-20-O-ester of 4-nitro-3-trifluoromethylphenoxyacetic acid

The mixture of homo-camptothecin (15 mg, 0.043 mmol), 4-nitro-3-trifluoromethylphenoxyacetic acid (20 mg, 0.075 mmol), EDCI (28 mg, 0.15 mmol), DMAP (2 mg, 0.02 mmol) and dichloromethane (3 ml) is stirred at room temperature for 20 h, then dichloromethane (20 ml) is added to the solution. The organic layer is washed with water (20 ml), saturated NaHCO₃ aqueous solution (10 ml) and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-4-nitro-3-trifluoromethylphenoxyacetic acid.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10, 11-methylenedioxy hCPT;

9-acetyloxy-10,11-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 27

This example explains how to prepare unsubstituted and substituted homo-camptothecin-20-O-esters of estra-1,3,5(10)-trien-17-one-3-oxyacetic acid.

A. Homo-camptothecin-20-O-ester of Estra-1,3,5(10)-trien-17-one-3-oxyacetic Acid Synthesis of Estra-1,3,5(10)-trien-17-one-3-oxyacetic Acid

The mixture of estrone (540 mg, 2.0 mmol), potassium carbonate (910 mg, 6.6 mmol), ethyl bromoacetate (500 mg, 3.0 mmol), and acetone (25 ml) was refluxed for 6 h. After cooling, the mixture was filtered to remove potassium ′carbonate. The filtrate was concentrated under reduced pressure. To this residue, 10 ml dioxane and 14 ml 5% sodium hydroxide solution ere added. After the mixture was stirred at room temperature overnight, it was acidified with concentrated hydrochloric acid to pH 2, and then extracted three times with ethyl acetate (15 ml each). The organic phases were combined, washed with water and brine, dried over magnesium sulfate, filtered, and then evaporated in vacuo. The residue was recrystallized from ethanol to give 246 mg estra-1,3,5(10)-trien-17-one-3-oxyacetic acid as white crystals, mp 205-208° C.

Synthesis of Homo-camptothecin-20-O-ester of Estra-1,3,5(10)-trien-17-one-3-oxyacetic Acid

The mixture of homo-camptothecin (10 mg, 0.029 mmol), estra-1,3,5(10)-trien-17-one-3-oxyacetic acid (20 mg, 0.061 mmol), EDCI (28 mg, 0.15 mmol), DMAP (2 mg, 0.02 mmol) and dichloromethane (3 ml) are stirred at room temperature for 20 h, then dichloromethane (20 ml) is added to the solution. The organic layer is washed with water (20 ml), saturated NaHCO₃ aqueous solution (10 ml), and brine (20 ml), and then dried over MgSO₄. After the solvent is removed under reduced pressure, the resulting solid is separated by column chromatography (eluent: CHCl₃: CH₃OH 9:1) to afford homo-camptothecin-20-O-2,6-dichloro-4-fluorophenoxyacetate.

B. By substituting other homo-camptothecin analogues for homo-camptothecin (hCPT) in part A of this example other compounds of this invention are prepared. In naming homo-camptothecin analogues, the standard numbering system for homo-camptothecin will be employed with “hCPT” being used as an abbreviation for homo-camptothecin. Other homo-camptothecin analogues include the following:

10,11-methylenedioxy hCPT;

9-nitro hCPT;

9-amino hCPT;

9-amino-10-hydroxy hCPT;

9-methylamino hCPT;

9-dimethylamino hCPT;

9-dimethylaminomethyl-10-,hydroxy hCPT;

9-piperidino hCPT;

9-morpholino hCPT

7-ethyl-10-[4-(1-piperidino)-1-piperidino]carbonyloxy)-hCPT;

7-t-butyldimethylsilyl hCPT;

7-t-butyldimethylsilyl-10-hydroxy hCPT;

9-nitro-10,11-methylenedioxy hCPT;

9-amino-10,11-methylenedioxy hCPT;

9-methyl-10,11-methylenedioxy hCPT;

9-chloro-10,11-methylenedioxy hCPT;

9-cyano-10,11-methylenedioxy hCPT;

9-acetyloxy-10,1-methylenedioxy hCPT;

9-acetylamino-10,11-methylenedioxy hCPT;

9-aminomethyl-10-hydroxy hCPT;

9-methylaminomethyl-10-hydroxy hCPT;

9-dimethylaminomethyl-10-hydroxy hCPT;

9-(2-hydroxyethyl) aminomethyl-10-hydroxy hCPT;

9-morpholinomethyl-10-hydroxy hCPT;

7-ethyl-10-hydroxy hCPT;

7-ethyl-10-acetyloxy hCPT;

7-methyl-10-aminocarbonyloxy hCPT;

7-n-propyl-10-piperidinocarbonyloxy hCPT;

7-ethyl-10-(2-dimethylamino) ethyl hCPT; and the like.

### Example 28

This example provides directions for growing cells and testing compounds of the invention for their effect on the growth of the cells. All cells are purchased from DCTDC Tumor Repository, NCI, NIH.

Cell Colony Formation Assay

Five hundred cells (VM46) were plated in 60 mm Petri dishes containing 2.7 ml of medium (modified McCoy's 5a medium containing 10% bovine calf serum and 100 units/ml penicillin and 100 mg/ml streptomycin). The cells were incubated in a CO₂ incubator at 37° C. for 5 hours for attachment to the bottom of Petri dishes. Drugs were made fresh in medium at ten times the final concentration, and then 0.3 ml of this stock solution was added to the 2.7 ml of medium in the dish. The cells were then incubated with drugs for 72 hours at 37° C. At the end of the incubation the drug-containing media were decanted, the dishes were rinsed with 4 ml of Hank's Balance Salt Solution (HBSS), 5 ml of fresh medium is added, and the dishes are returned to the incubator for colony formation. The cell colonies were counted using colony counter after incubation for 8 days for VM46 cells. Cell survival (%) is calculated, as shown in Table I.

Values of ID50 (the drug concentration producing 50% inhibition of colony formation) may be determined for each tested compound.

TABLE I

| Compounds | Values of ID 50 (nM) |
|---|---|
| homo-campothecin | 4 |
| 021229 | 2 |
| 1158 | 1.5 |
| 1160 | 1.5 |
| 1164 | 3.5 |

### Example 29

This example provides directions for performing in vivo toxicity tests of the compounds of the invention on C3H/HeJ mice.

Acute toxicities of the compounds of this invention are evaluated on C3H/HeJ mice (body weight 18-22 g). The MTD40 (maximum tolerated dose at day 40) values are determined by the standard procedure described by Gad and Chengelis (see, for example, “Acute Toxicology Testing,” 2^{nd} Ed., Shayne O. Gad and Christopher P. Chengelis, pp. 186-195 (Academic Press)). In the consecutive type studies, 2 mice are dosed at low and moderate doses of 40 and 100 mg/kg. If no severe and irreversible toxicity (euthanasia is required) occurs at these doses, a new pair of animals is initiated at 180 mg/kg, which is 1.8 times higher than 100 mg/kg. Sequential dosages (about 3 doses on 3 pairs of animals, i.e. 2 mice for each drug dose) are increased by a factor of 1.8 until severe and irreversible toxicity (euthanasia is required) occurs. Then another pair of animals is initiated at the highest nonlethal dosage, and successive dosages are increased by a factor of 1.15. The result of this exercise is two dosages, one apparently nonlethal and the other lethal if severe and irreversible toxicity occurs and euthanasia is required, separated by a factor of 1.15. Six mice are dosed at each dosage. If no severe and irreversible toxicity occurred at the lower dosage and at least one with severe and irreversible toxicity occurred at the higher dose, then the lower dose is considered to be the MTD. The compounds of this invention are administered to C3H/HeJ mice by intraperitoneal injection. Drug toxicity is evaluated on mice checked daily for 45 days. The toxicity parameters reported are the MTD40. The MTD is defined as the highest dose causing no severe irreversible toxicity in one treatment group, but at least one animal exhibiting severe and irreversible toxicity and being euthanized at the next higher dose.

### Example 30

This example provides directions for performing in vivo efficacy tests of the compounds of the invention on C3H/HeJ mice bearing MTG-B tumors.

Studies on the compounds of this invention are performed on C3H/HeJ mice bearing MTG-B tumors. The tumors grow exponentially following implantation into the flanks of the mice and reached a diameter of 8 mm (268.08 mm³) by day 7 to 10. Treatment is initiated at that time, with the first day of treatment designated as day 0 for calculation and plots. The mice are injected i.p. with three drug dose levels (1/3, 1/2, 1 5 MTD) using both a single injection and the schedule of Q2D 5 3 (every 2 days for a total of 3 treatments at 1/3 MTD). Control groups of mice bearings8 mm diameter tumors are treated with vehicle alone. After drug treatment, the mice are observed twice a day. When a tumor reaches 1.5 g, the mouse bearing the tumor is euthanized. Surviving days measured from day 0 for mice treated with anticancer drugs (T) and surviving days measured from day 0 for control mice (C) are recorded. Tumor growth inhibition values (T/C %) are calculated using the formula T/C %=(surviving days of mice treated with an anticancer drug T/surviving days of control mice C) 5 100%.

Tumor sizes may be measured by caliper every day. Daily measurement (mm) of solid tumor (length L and width W) in two dimensions is used to calculate the tumor weight [tumor weight=(length 5 width²)/2] based on the interchangeable value of 1mm³=1 mg. Tumor growth delay (T−C value) is determined by calculation of the median time (in days) required for the treatment group and control group tumors to reach 1,000 mg. Tumor doubling time (Td) is measured, and tumor cell kill is calculated by the formula of log cell kill=(T−C value)/(3.32 5 Td). Regression effects after treatment may be observed and recorded (a complete regression: a regression-below limit of palpation; a partial regression: a regression of more than 50% reduction in tumor mass).

### Example 31

This example provides guidance for determining the hydrolysis kinetics of the lactone ring (E) of homo-camptothecin derivatives in the presence of different blood components. A quantitative C₁₈ reversed-phase high-performance liquid chromatography (HPLC) assay can be employed. A description is found at the following references:

J. Med. Chem. 2000, 43, 3970-3980;

Anal. Biochem. 1993, 212, 285-287; and

Biochemistry 1994, 33, 10325-10336.

See also J. Med. Chem. 1998, 41, 31-37.

### Example 32

This example provides guidance for determining the inhibition of topoisomerase I. This procedure is an intact cell assay and is a modification of a published procedure found at Cancer Res. 1986, 46, 2021-2026. A more recent publication can be found at J. Med. Chem. 1993, 36 2689-2700 at 2699. Here the modification of the previous procedure is used to quantitate the amount of topoisomerase I mediated DNA cleavage in intact cells. The DNA of HL-60 cells growing in culture is labeled by [³H] thymidine incorporation. The cells are exposed to compounds to be tested and lysed, and the protein is precipitated. Radioactive DNA in cleavable complex formation with topoisomerase I coprecipitates with the protein. The amount of cleavable complex formation is quantitated by counting the pellet with a liquid scintillation counter.

The subject matter claimed is:

1. A compound of the formula 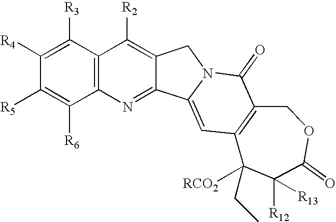
wherein R is R₁—O—(CH₂)ₘ—, m is an integer of 1-10;
R₁ is lower alkyl,
phenyl optionally substituted with from one to five substituents independently selected from the group consisting of halo, lower alkyl, lower alkoxy, hydroxy, cyano, nitro, amino, halogenated lower alkyl, halogenated lower alkoxy, formyl, lower alkyl carbonyl, hydroxycarbonyl, lower alkylcarbonyloxy, benzyloxy, optionally substituted piperidino, lower alkoxycarbonyl, and lower alkylcarbonylamino,
cycloalkyl of 3-7 carbons, optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino,
a fused, 2-, 3-, or 4-ring heterocyclic radical optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino,
1- or 2-naphthyl optionally substituted with from one to four substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino, or
a 5- or 6-membered heterocyclic ring containing one or two nitrogen atoms, which ring is optionally substituted with one or two substituents selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino;
R₂ is hydrogen, halo, lower alkyl, lower alkoxy, hydroxy, RC(O)O (R is defined hereinbefore), cyano, nitro, amino, halogenated lower alkyl, halogenated lower alkoxy, hydroxycarbonyl, formyl, lower alkoxycarbonyl, tri lower alkylsilyl, lower alkylcarbonyloxy, lower alkylcarbonylamino, lower alkylcarbonyloxymethyl, substituted vinyl, 1-hydroxy-2-nitroethyl, alkoxycarbonylethyl, aminocarbonyl, mono- or di-alkylcarbonyl, alkylcarbonyloxymethyl, benzoylmethyl, benzylcarbonyloxymethyl, or mono- or di lower alkoxymethyl;
R₃ is hydrogen, halo, lower alkyl, lower alkoxy, hydroxy, RC(O)O (R is defined hereinbefore) cyano, nitro, amino, halogenated lower alkyl, halogenated lower alkoxy, hydroxycarbonyl, formyl, lower alkoxycarbonyl, CH₂NR₇R₈ (where each of R₇ and R₈ is independently H—, alkyl of 1-6 carbons, optionally substituted phenyl, hydroxy lower alkyl, amino lower alkyl, or mono- or dialkylamino lower alkyl, or R₇ and R₈ taken together with —N-represent a cyclic amino-), CH₂R₉ (where R₉ is lower alkoxy, CN, amino lower alkoxy, mono- or di-lower alkylamino lower alkoxy, lower alkylthio, amino lower alkylthio, or mono- or di-lower alkylamino lower alkylthio), or NR₁₀R₁₁ (where each of R₁₀ and R₁₁ is independently hydrogen, lower alkyl, phenyl, hydroxy lower alkyl, amino lower alkyl, or mono- or di-lower alkyl, or R₁₀ and R₁₁ taken together with —N— represent a cyclic amino), dialkylamino alkyl, lower alkylcarbonyloxy, or lower alkylcarbonylamino;
R₄ is hydrogen, halo, lower alkyl, lower alkoxy, hydroxy, RC(O)O (R is defined hereinbefore) cyano, nitro, amino, amino lower alkyl, halogenated lower alkyl, halogenated lower alkoxy, hydroxycarbonyl, formyl, lower alkoxycarbonyl, carbamoyloxy, lower alkylcarbonyloxy, or lower alkylcarbonylamino, or R₄ together with R₅ is methylenedioxy or ethylenedioxy;
R₅ is hydrogen, halo, lower alkyl, lower alkoxy, hydroxy, RC(O)O (R is defined hereinbefore) cyano, nitro, amino, halogenated lower alkyl, halogenated lower alkoxy, hydroxycarbonyl, formyl, lower alkoxycarbonyl, lower alkylcarbonyloxy, or lower alkylcarbonylamino or R₅ together with R₄ is methylenedioxy or ethylenedioxy;
R₆ is hydrogen, halo, lower alkyl, lower alkoxy, hydroxy, RC(O)O (R is defined hereinbefore) cyano, nitro, amino, halogenated lower alkyl, halogenated lower alkoxy, hydroxycarbonyl, formyl, lower alkoxycarbonyl, lower alkylcarbonyloxy, or lower alkylcarbonylamino; and
R₁₂ and R₁₃ are independently hydrogen, lower alkyl, cyano, hydroxycarbonyl, lower alkoxycarbonyl, cycloalkyl, alkylcarbonylamino, 1-napthyl, 2-napthyl or phenyl.

2. The compound of claim 1, wherein m is 1; each of R₂ through R₆, R₁₂, and R₁₃ is H; and R₁ is phenyl optionally substituted with one to three substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, and benzyloxy.

3. The compound of claim 2, wherein R₁ is phenyl optionally substituted with one to three substituents independently selected from lower alkyl, halo, halogenated lower alkoxy, and lower alkoxy.

4. The compound of claim 3, wherein R₁ is phenyl optionally substituted with one to three halo substituents.

5. The compound of claim 4, wherein R₁ is phenyl.

6. The compound of claim 4, wherein R₁ is 4-halophenyl.

7. The compound of claim 4, wherein R₁ is 3-chlorophenyl or 2-chlorophenyl.

8. The compound of claim 4, wherein R₁ is 2,4-dichlorophenyl.

9. The compound of claim 4, wherein R₁ is 4-fluorophenyl, 4-bromophenyl, or 4-iodophenyl.

10. The compound of claim 4, wherein R₁ is 2,3-dichlorophenyl.

11. The compound of claim 2, wherein R₁ is 4-trifluoromethoxyphenyl.

12. The compound of claim 3, wherein R₁ is phenyl substituted with one or two lower alkyl substituents.

13. The compound of claim 12, wherein R₁ is 4-methylphenyl.

14. The compound of claim 12, wherein R₁ is 2,4-dialkyl-substituted phenyl.

15. The compound of claim 2, wherein R₁ is phenyl substituted with one or two lower alkoxy substituents.

16. The compound of claim 2, wherein R₁ is 4-methoxyphenyl.

17. The compound of claim 1, wherein m is 2 and R₂ is a 5- or 6-membered heterocyclic ring.

18. The compound of claim 17, wherein R₁ is 1-piperidine.

19. The compound of claim 1, wherein m is 1; each of R₂ through R₆, R₁₂, and R₁₃ is H; and R₁ is a fused, 2-ring heterocyclic system.

20. The compound of claim 1, wherein m is 1; each of R₂ through R₆, R₁₂, and R₁₃ is H; and R₁ is 1- or 2-naphthyl optionally substituted with from one to four substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy and lower alkylcarbonylamino.

21. The compound of claim 20, wherein R₁ is 2-naphthyl.

22. The compound of claim 1, wherein m is an integer of 2-4; each of R₂ through R₆, R₁₂, and R₁₃ is H; and R₁ is phenyl optionally substituted with from one to five substituents independently selected from the group consisting of halo, lower alkyl, lower alkoxy, hydroxy, cyano, nitro, amino, halogenated lower alkyl, halogenated lower alkoxy, carbonyl, hydroxycarbonyl, lower alkoxycarbonyl, benzyloxy, lower alkylcarbonyloxy and lower alkylcarbonylamino.

23. The compound of claim 1, wherein each of R₆, R₁₂, and R₁₃ is hydrogen

24. The compound of claim 23, wherein R₄ and R₅ together are methylenedioxy.

25. The compound of claim 24, wherein R₂ is hydrogen.

26. The compound of claim 25, wherein R₃ is nitro, amino, methyl, chloro, cyano, acetoxy, or acetylamino.

27. The compound of claim 23, wherein R₅ is hydrogen.

28. The compound of claim 27, wherein R₃ is hydrogen; R₂ is (3-chloro-n-propyl)dimethylsilyl, tert-butyldimethylsilyl, acetoxymethyl, cyano, formylethenyl, ethoxycarbonyl-ethenyl, cyanoethenyl, 2,2-dicyanoethenyl(2-cyano-2-ethoxycarbony)ethenyl, ethoxycarbon ethyl, methyl, ethyl, or n-propyl; and R₄ is hydroxy, acetoxy, amino, nitro, cyano, chloro, bromo, fluoro, lower alkyl, higher alkyl, lower alkoxy, carbamoyloxy, or formyl.

29. The compound of claim 1, wherein R₂ is ethyl, R₄ is carbamoyloxy, and each of R₃, R₅, R₆, R₁₂, and R₁₃, is hydrogen.

30. The compound of claim 29, wherein carbamoyloxy is 1-pyrazinylcarbonyloxy, 4(-i-propylaminocarbonylmethyl)-1-pyrazinyl-carbonyloxy, or [4-(1-piperidino)-1-piperidino]-carbonyloxy.

31. The compound of claim 1, wherein each of R₂, R₅, R₆, R₁₂, and R₁₃ is hydrogen; R₃ is amino, nitro, cyano, halo, OH, lower alkylamino, di-lower alkylamino, lower alkyl, lower alkoxy, 1-piperidino, 1-mopholino, aminomethyl, lower alkylaminomethyl, cycloalkylaminomethyl, di-lower alkylaminomethyl, cyclic aminomethyl, acetoxy, acetylamino, lower alkoxymethyl, omega-hydroxy lower alkylaminomethyl, cyanomethyl; and R₄ is hydroxy, acetoxy, cyano, nitro, amino, halo, formyl, lower alkoxy, carbamoyloxy.

32. The compound of claim 1, wherein each of R₂, R₃, R₅, R₆, R₁₂, and R₁₃ is hydrogen and R₄ is —OC(O)Alkyl₁₋₂₀.

33. The compound of claim 1, wherein m is 1.

34. The compound of claim 1, wherein R₂ is hydrogen;
R₃ is CH₂NR₇R₈ (where each of R₇ and R₈ is independently H—, alkyl of 1-6 carbons, optionally substituted phenyl, hydroxy lower alkyl, amino lower alkyl, or mono- or dialkylamino lower alky, or R₇ and R₈ taken together with —N— represent a cyclic amino-), NR₁₀R₁₁ (where each of R₁₀ and R₁₁ is independently hydrogen, lower alkyl, phenyl, hydroxy lower alkyl, amino lower alkyl, or mono- or di-lower alkyl, or R₁₀ and R₁₁ taken together with —N— represent a cyclic amino), or dialkylamino alkyl;
R₄ is lower alkoxy, hydroxy, halogenated lower alkyl, halogenated lower alkoxy, hydroxycarbonyl, formyl, lower alkoxycarbonyl, carbamoyloxy, lower alkylcarbonyloxy, or R₄ together with R₅ is methylenedioxy or ethylenedioxy;
R₅ is hydrogen or together with R₄ is methylenedioxy or ethylenedioxy;
R₆ is hydrogen.

35. The compound of claim 34, wherein R₃ is CH₂NR₇R₈ (where each of R₇ and R₈ is lower alkyl), R₄ is hydroxy, alkoxy or alkylcarbonyloxy and R₁₂ and R₁₃ are hydrogen.

36. The compound of claim 35, wherein m is 1 and R₃ is CH₂N(CH₃)₂ and R₄ is hydroxy.

37. The compound of claim 1, wherein R₂ is hydrogen, lower alkyl, or halogenated lower alkyl;
R₃ is hydrogen or lower alkyl;
R₄ is lower alkoxy, hydroxy, halogenated lower alkoxy, carbamoyloxy, lower alkylcarbonyloxy, or R₄ together with R₅ is methylenedioxy;
R₅ is hydrogen or together with R₄ is methylenedioxy;
R₆ is hydrogen.

38. The compound of claim 37, wherein R₃ is hydrogen, R₅ is hydrogen, R₄ is carbamoyloxy and R₁₂ and R₁₃ are hydrogen.

39. The compound of claim 38, wherein R₂ is lower alkyl and R₄ is 4-(1-piperidino)-1-piperidinocarbonyloxy.

40. The compound of claim 39, wherein m is 1 and R₂ is ethyl.

41. The compound of claim 1, wherein
R₂ is lower alkyl;
R₃ is hydrogen;
R₄ is hydroxy, lower alkoxy, halogenated lower alkoxy, hydroxycarbonyl, formyl, lower alkoxycarbonyl, carbamoyloxy, lower alkylcarbonyloxy;
R₅ is hydrogen;
R₆ is hydrogen.

42. The compound of claim 41, wherein R₂ is ethyl, R₄ is hydroxy and R₁₂ and R₁₃ are hydrogen.

43. The compound of claim 42, wherein m is 1.

44. The compound of claim 1, wherein each of R₂, R₄, R₅, and R₆ is hydrogen and R₃ is amino or nitro.

45. The compound of claim 44, wherein R₃ is amino and R₁₂ and R₁₃ are hydrogen.

46. The compound of claim 45, wherein m is 1.

47. The compound of claim 44, wherein R3 is nitro.

48. The compound of claim 47, wherein m is 1.

49. The compound of claim 1, wherein
R₂ is tri-lower alkylsilyl;
R₃ is hydrogen;
R₄ is hydroxy, lower alkoxy, halogenated lower alkoxy, hydroxycarbonyl, formyl, lower alkoxycarbonyl, carbamoyloxy, lower alkylcarbonyloxy;
R₅ is hydrogen;
R₆ is hydrogen.

50. The compound of claim 49, wherein R₂ is t-butyldimethylsilyl, R₄ is hydroxy, and R₁₂ and R₁₃ are hydrogen.

51. The compound of claim 50, wherein m is 1.

52. A pharmaceutical composition useful for treating cancer in a warm-blooded animal, which composition comprises compound as defined in claim 1 in combination with a pharmaceutically acceptable excipient.

53. The pharmaceutical composition of claim 52 suitable for oral administration.

54. The pharmaceutical composition of claim 52 suitable for IV administration.

55. The pharmaceutical composition of claim 52 suitable for IM administration.

56. A method for treating cancer in a warm-blooded animal, which method comprises administering a therapeutically effective amount of a compound as defined in claim 1.

57. The method of claim 56, wherein the compound is administered orally.

58. The method of claim 56, wherein the compound is administered IV.

59. The method of claim 56, wherein the compound is administered parenterally.

60. A process of preparing a compound of claim 1, which comprises reacting
(a) a compound of the formula, R—C(O)X, wherein R is R₁—O—(CH₂)ₘ, R₁ and m are defined as in claim 1, and X is hydroxy, chloride, or R—C(O)—O (where R is defined hereinbefore) with
(b) a compound of the formula, 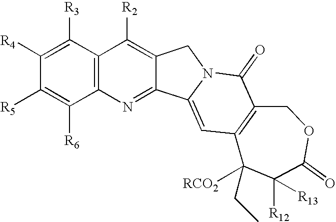
wherein R₂, R₄, R₅, R₆, R₁₂ and R₁₃ are defined in claim 1.

61. The process of claim 60, wherein the reacting takes place in the presence of the coupling agent 1-(3-dimethylaminopropyl)-3-ethylcarbodiimide hydrochloride and the catalytic agent 4-(dimethylamino) pyridine.

62. A compound of the formula 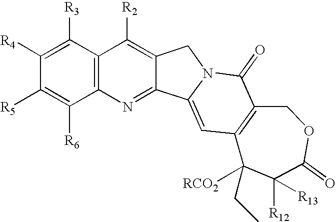
wherein R is R₁—O—(CH₂)ₘ—, m is an integer of 1-10;
R₁ is lower alkyl,
phenyl optionally substituted with from one to five substituents independently selected from the group consisting of halo, lower alkyl, lower alkoxy, hydroxy, cyano, nitro, amino, halogenated lower alkyl, halogenated lower alkoxy, formyl, lower alkyl carbonyl, hydroxycarbonyl, lower alkylcarbonyloxy, benzyloxy, optionally substituted piperidino, lower alkoxycarbonyl, and lower alkylcarbonylamino,
cycloalkyl of 3-7 carbons, optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino,
a fused, 2-, 3-, or 4-ring heterocyclic radical optionally substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino,
1- or 2-naphthyl optionally substituted with from one to four substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino, or
a 5- or 6-membered heterocyclic ring containing one or two nitrogen atoms, which ring is optionally substituted with one or two substituents selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino;
R₁₂ and R₁₃ are independently hydrogen, lower alkyl, cyano, hydroxycarbonyl, lower alkoxycarbonyl, cycloalkyl, alkylcarbonylamino, 1-napthyl, 2-napthyl or phenyl; and
R₂ is —CH₂NR₇R₈ (where R₇ and R₈ taken together with —N— represent a cyclic amino) when R₃ and R₆ are H and R₄ and R₅ taken together represent ethylenedioxy; or
R₂ is butoxyiminomethyl when each of R₃, R₄, R₅ and R₆ is H; or
R₃ is trialkylsilylethylene when each of R₂, R₄, R₅, and R₆ is H; or
R₂ and R₃ together represent —CH(NR₁₂R₁₃)—CH₂—CH₂— when R₄ is lower alkyl, R₅ is halo, and R₆ is H, wherein R₁₂ and R₁₃ each is independently H or lower alkyl.

63. A pharmaceutical composition useful for treating cancer in a warm-blooded animal, which composition comprises compound as defined in claim 62 in combination with a pharmaceutically acceptable excipient.

64. A method for treating cancer in a warm-blooded animal, which method comprises administering a therapeutically effective amount of a compound as defined in claim 62.
